(19) United States
(12) Patent Application Publication
(10) Pub. No.: US 2022/0305129 A1
(43) Pub. Date: Sep. 29, 2022

(54) CONJUGATES OF HETEROAROMATIC NITROGEN-COMPRISING COMPOUNDS

(71) Applicant: Ascendis Pharma A/S, Hellerup (DK)

(72) Inventors: Nicola BISEK, Heidelberg (DE); Samuel WEISBROD, Heidelberg (DE)

(21) Appl. No.: 17/596,833

(22) PCT Filed: Jun. 19, 2020
(86) PCT No.: PCT/EP2020/067152

§ 371 Date: Dec. 20, 2021

(30) Foreign Application Priority Data
Jun. 21, 2019 (EP) 19181795.6

(51) Int. Cl. A61K 47/60; A61K 47/69

(52) U.S. Cl. CPC A61K 47/60; A61K 47/6903; A61K 47/6939; A61K 31/728

(57) ABSTRACT

The present invention relates to conjugates of π-electron-pair-donating heteroaromatic nitrogen-comprising drugs and pharmaceutically acceptable salts thereof, pharmaceutical compositions comprising said conjugates and the use of said conjugates as medicaments.

The present invention relates to conjugates of π-electron-pair-donating heteroaromatic nitrogen-comprising drugs and pharmaceutically acceptable salts thereof, pharmaceutical compositions comprising said conjugates and the use of said conjugates as medicaments.

To improve physicochemical or pharmacokinetic properties, such as the in vivo circulation half-life of drugs, such drugs can be conjugated to a carrier, such as a polymer. Typically, polymers in drug delivery are either used in a non-covalent complexation of the drug and polymer, embedding of drug in a polymer or by covalent conjugation of the drug to a polymeric moiety.

However, the non-covalent approach requires a highly efficient drug encapsulation to prevent uncontrolled, burst-type release of the drug due to the disintegration of the drug-polymer complex after administration. Restraining the diffusion of an unbound, water-soluble drug molecule requires strong van der Waals contacts, frequently mediated through hydrophobic moieties and charged moieties for electrostatic binding. Many conformationally sensitive drugs, such as proteins or peptides, are rendered dysfunctional during the complexation process and/or during subsequent storage of the non-covalently bound drug.

Alternatively, a drug may be covalently conjugated to a polymeric moiety via a stable linker or a reversible linker from which the drug is released. If the drug is stably conjugated to the polymeric moiety, such conjugate needs to exhibit sufficient residual activity to have a pharmaceutical effect and thus the conjugate is constantly in an active form.

One advantage of conjugating a drug to a polymeric moiety through a reversible linker is that no residual activity of the conjugate is needed, because the drug exhibits its pharmacological effect upon release from the conjugate. A conjugate may exhibit no or little drug activity, i.e. the conjugate is pharmacologically inactive. This approach is applied to all classes of molecules, from so-called small molecules, through natural products up to large proteins. The drug of such a conjugate may be released by enzymatic or non-enzymatic cleavage of the linkage between the polymeric moiety and the drug moiety or by a combination of both. However, enzyme-dependence is usually less preferred, because enzyme levels may vary significantly between patients what makes the correct dosing difficult.

WO 2005/099768 A2, WO 2009/095479 A2 and WO 2016/196124 A2 disclose carrier-linked prodrugs whereby drug moieties are reversibly connected to transient linkers via amines such as aliphatic amines, by formation of for example, amide bonds. Such aliphatic amines consist of only hydrogen and alkyl substituents. WO 2011/012722 A1 discloses carrier-linked prodrugs, whereby the drug moieties are attached via their aromatic amines to reversible linkers through formation of amide bonds. Such aromatic amines comprise an aromatic ring to which the nitrogen atom of the amine is attached, meaning that the nitrogen atom of aromatic amines is not part of the aromatic ring system. Although the beforementioned patent applications report the ability of converting drug moieties that comprise aliphatic and aromatic amines into conjugates, they do not explore the ability of explicitly employing n-electron-pair-donating heteroaromatic nitrogens of drug molecules as linkage points to reversible linkers. Such moieties are usually good leaving groups, so the expectation is that any polymer conjugated to such moiety via the aforementioned linkers may be cleaved off too rapidly to provide any meaningful half-life extension. Therefore, there is still a need for conjugates, in which the linker attachment takes place at the π-electron-pair-donating heteroaromatic nitrogens.

CN106478596A discloses axitinib prodrugs that were designed to increase the water-solubility and lipophilicity of axitinib, whereby functionalities such as methyl acetate, methyl hydrogen carbonate and methyl dihydrogen phosphate derivatives were attached to the nitrogen atoms of axitinib, while lauric acid was attached to the methyl group of the amide functionality of axitinib. However, CN106478596A does not disclose for example how to avoid the liberation of potentially toxic side-products which may result by the cleavage of the functionalities that were attached to the nitrogen atoms of axitinib.

It is thus an object of the present invention to at least partially overcome the shortcomings described above.

This object is achieved with a conjugate or a pharmaceutically acceptable salt thereof comprising at least one moiety -D conjugated via at least one moiety -L¹-L²- to at least one moiety Z, wherein a moiety -L¹- is conjugated to a π-electron-pair-donating heteroaromatic N of a moiety -D and wherein the linkage between -D and -L¹- is reversible and wherein a moiety -L²- is conjugated to Z, wherein
each -D is independently a π-electron-pair-donating heteroaromatic N-comprising moiety of a drug D-H;
each -L²- is independently a single bond or a spacer moiety;
each Z is independently a polymeric moiety or a C₈₋₂₄ alkyl;
each -L¹- is independently a linker moiety of formula (I):

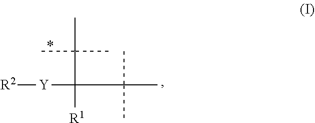

wherein
   the dashed line marked with an asterisk indicates the attachment to -L²-;
   the unmarked dashed line indicates the attachment to the π-electron-pair-donating heteroaromatic N of -D;
   —Y— is selected from the group consisting of —N(R³)—, —O— and —S—;
   —R¹, —R2 and —R³ are independently selected from the group consisting of —H, -T, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R4, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵)—, —OC(OR⁵)(R^{5a})—, —N(R⁵)C(O)N(R^{5a})— and —OC(O)N(R⁵)—;
      each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each T is independently optionally substituted with one or more —R⁴, which are the same or different;
      wherein —R⁴, —R⁵ and —R^{5a} are independently selected from the group consisting of —H and C₁₋₆ alkyl; wherein C₁₋₆ alkyl is optionally substituted with one or more halogen, which are the same or different; and
each -L¹- is substituted with -L²- and optionally further substituted.

It was surprisingly found that the reversible linker moiety -L¹- has advantageous properties, such as for example avoiding the liberation of potentially toxic side-products which may result after cleavage of the drug.

Within the meaning of the present invention the terms are used as follows.

As used herein, the term “a π-electron-pair-donating heteroaromatic N-comprising moiety” refers to the moiety which after cleavage of the linkage between -D and -L¹- results in a drug D-H and wherein the drug moiety -D and analogously the corresponding D-H comprises at least one, such as one, two, three, four, five, six, seven, eight, nine or ten heteroaromatic nitrogen atoms that donate a π-electron pair to the aromatic π-system. Examples of chemical structures comprising such heteroaromatic nitrogens that donate a π-electron pair to the aromatic π-system include, but are not limited to, pyrrole, pyrazole, imidazole, isoindazole, indole, indazole, purine, tetrazole, triazole and carbazole. For example, in the imidazole ring below the heteroaromatic nitrogen which donates a π-electron pair to the aromatic π-system is marked with “#”:

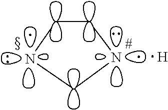

The π-electron-pair-donating heteroaromatic nitrogen atoms do not comprise heteroaromatic nitrogen atoms which only donate one π-electron (i.e. not a pair of π-electrons) to the aromatic π-system, such as for example the nitrogen that is marked with “§ ” in the abovementioned imidazole ring structure. The drug D-H may exist in one or more tautomeric forms, such as with one hydrogen atom moving between at least two heteroaromatic nitrogen atoms. In all such cases, the linker moiety is covalently and reversibly attached at a heteroaromatic nitrogen that donates a π-electron pair to the aromatic π-system.

As used herein, the term “drug” refers to a substance used in the treatment, cure, prevention or diagnosis of a disease or used to otherwise enhance physical or mental well-being of a patient. If a drug is conjugated to another moiety, the moiety of the resulting product that originated from the drug is referred to as “drug moiety”.

As used herein, the term “moiety” means a part of a molecule, which lacks one or more atom(s) compared to the corresponding reagent. If, for example, a reagent of the formula “H—X—H” reacts with another reagent and becomes part of the reaction product, the corresponding moiety of the reaction product has the structure “H—X—” or “—X—”, whereas each “—” indicates attachment to another moiety. Accordingly, a drug moiety is released from a reversible linkage as a drug.

It is understood that if a chemical structure of a group of atoms is provided which group of atoms is attached to two moieties or is interrupting a moiety, said sequence or chemical structure can be attached to the two moieties in either orientation, unless explicitly stated otherwise. For example, a moiety “—C(O)N(R^{x})—” can be attached to two moieties or interrupting a moiety either as “—C(O)N(R^{x})—” or as “—N(R^{x})C(O)—”. Similarly, a moiety:

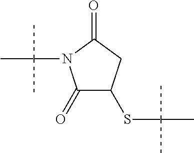

can be attached to two moieties or can interrupt a moiety either as

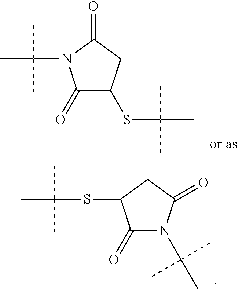

As used herein, the term “reagent” means a chemical compound, which comprises at least one functional group for reaction with the functional group of another chemical compound or drug. It is understood that a drug comprising a functional group is also a reagent.

It is recognized by one of ordinary skill in the art that the conjugates of the present invention are prodrugs. As used herein, the term “prodrug” refers to a drug moiety, that is reversibly and covalently conjugated to a polymeric moiety, such as Z, through at least one -L¹-L²- moiety. A prodrug releases the reversibly and covalently bound drug moiety -D in the form of its corresponding drug D-H. In other words, a prodrug is a conjugate comprising a drug moiety, which is covalently and reversibly conjugated to a polymeric moiety via at least one -L¹-L²- moiety. Such prodrugs or conjugates release the formerly conjugated drug moiety in the form of a free drug.

As used herein, the term “reversible linkage” or “biodegradable linkage” is a linkage that is cleavable, in the absence of enzymes under physiological conditions, which are aqueous buffer at pH 7.4 and 37° C., with a half-life ranging from one hour to six months, such as from one hour to four months, such as from one hour to three months, from one hour to two months or from one hour to one month. It is understood, however, that a reversible linkage may also be cleavable at other conditions, such as for example at a different pH or at a different temperature with a half-life ranging from one hour to six months, but that a test for determining reversibility is performed in the above-described physiological conditions (aqueous buffer, pH 7.4, 37° C.). Accordingly, a “stable linkage” is a linkage having a half-life under physiological conditions of more than six months.

As used herein, the term “reversible linker moiety” is a moiety which is covalently conjugated to a drug moiety through a reversible linkage and which is also covalently conjugated to a moiety Z via a moiety -L²-. In certain embodiments, the linkage between Z and -L²- is a stable linkage.

As used herein, the term “about” in combination with a numerical value is used to indicate a range ranging from and including the numerical value plus and minus no more than 10% of said numerical value, in certain embodiments, no more than 8% of said numerical value, in certain embodiments, no more than 5% of said numerical value and in certain embodiments, no more than 2% of said numerical value. For example, the phrase “about 200” is used to mean a range ranging from and including 200+/−10%, i.e. ranging from and including 180 to 220; in certain embodiments, 200+/−8%, i.e. ranging from and including 184 to 216; in certain embodiments, ranging from and including 200+/−5%, i.e. ranging from and including 190 to 210; and in certain embodiments 200+/−2%, i.e. ranging from and including 196 to 204. It is understood that a percentage given as “about 20%” does not mean “20%+/−10%”, i.e. ranging from and including 10 to 30%, but “about 20%” means ranging from and including 18 to 22%, i.e. plus and minus 10% of the numerical value which is 20.

As used herein, the term “C₁₋₄ alkyl” alone or in combination means a straight-chain or branched alkyl moiety having 1 to 4 carbon atoms. If present at the end of a molecule, examples of straight-chain or branched C₁₋₄ alkyl are methyl, ethyl, n-propyl, isopropyl, n-butyl, isobutyl, sec-butyl and tert-butyl. When two moieties of a molecule are linked by the C₁₋₄ alkyl, then examples for such C₁₋₄ alkyl groups are —CH₂—, —CH₂—CH₂—, —CH(CH₃)—, —CH₂—CH₂—CH₂—, —CH(C₂H₅)—, —C(CH₃)₂—. Each hydrogen of a C₁₋₄ alkyl carbon may optionally be replaced by a substituent as defined below. Optionally, a C₁₋₄ alkyl may be interrupted by one or more moieties as defined below.

As used herein, the term “C₁₋₆ alkyl” alone or in combination means a straight-chain or branched alkyl moiety having 1 to 6 carbon atoms. If present at the end of a molecule, examples of straight-chain and branched C₁₋₆ alkyl groups are methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. When two moieties of a molecule are linked by the C₁₋₆ alkyl group, then examples for such C₁₋₆ alkyl groups are —CH₂—, —CH₂—CH₂—, —CH(CH₃)—, —CH₂—CH₂—CH₂—, —CH(C₂H₅)— and —C(CH₃)₂—. Each hydrogen atom of a C₁₋₆ carbon may optionally be replaced by a substituent as defined below. Optionally, a C₁₋₆ alkyl may be interrupted by one or more moieties as defined below.

Accordingly, “C₁₋₁₀ alkyl”, “C₁₋₂₀ alkyl”, “C₈₋₂₄ alkyl” or “C₁₋₅₀ alkyl” means an alkyl chain having 1 to 10, 1 to 20, 8 to 24 or 1 to 50 carbon atoms, respectively, wherein each hydrogen atom of the C₁₋₁₀, C₁₋₂₀, C₈₋₂₄ or C₁₋₅₀ carbon may optionally be replaced by a substituent as defined below. Optionally, a C₁₋₁₀ alkyl, C₁₋₂₀ alkyl, C₈₋₂₄ alkyl or C₁₋₅₀ alkyl may be interrupted by one or more moieties as defined below.

As used herein, the term “C₂₋₆ alkenyl” alone or in combination means a straight-chain or branched hydrocarbon moiety comprising at least one carbon-carbon double bond having 2 to 6 carbon atoms. If present at the end of a molecule, examples are —CH═CH₂, —CH═CH—CH₃, —CH₂—CH═CH₂, —CH═CHCH₂—CH₃ and —CH═CH—CH═CH₂. When two moieties of a molecule are linked by the C₂₋₆ alkenyl group, then an example of such C₂₋₆ alkenyl is —CH═CH—. Each hydrogen atom of a C₂₋₆ alkenyl moiety may optionally be replaced by a substituent as defined below. Optionally, a C₂₋₆ alkenyl may be interrupted by one or more moieties as defined below.

Accordingly, the terms “C₂₋₁₀ alkenyl”, “C₂₋₂₀ alkenyl” or “C₂₋₅₀ alkenyl” alone or in combination mean a straight-chain or branched hydrocarbon moiety comprising at least one carbon-carbon double bond having 2 to 10, 2 to 20 or 2 to 50 carbon atoms, respectively. Each hydrogen atom of a C₂₋₁₀ alkenyl, C₂₋₂₀ alkenyl or C₂₋₅₀ alkenyl group may optionally be replaced by a substituent as defined below. Optionally, a C₂₋₁₀ alkenyl, C₂₋₂₀ alkenyl or C₂₋₅₀ alkenyl may be interrupted by one or more moieties as defined below.

As used herein, the term “C₂₋₆ alkynyl” alone or in combination means a straight-chain or branched hydrocarbon moiety comprising at least one carbon-carbon triple bond having 2 to 6 carbon atoms. If present at the end of a molecule, examples are —C≡CH, —CH₂—C≡CH, CH₂—CH₂—C≡CH and CH₂—C≡C—CH₃. When two moieties of a molecule are linked by the alkynyl group, then an example is —C≡C—. Each hydrogen atom of a C₂₋₆ alkynyl group may optionally be replaced by a substituent as defined below. Optionally, one or more double bond(s) may occur. Optionally, a C₂₋₆ alkynyl may be interrupted by one or more moieties as defined below.

Accordingly, as used herein, the term “C₂₋₁₀ alkynyl”, “C₂₋₂₀ alkynyl” and “C₂₋₅₀ alkynyl” alone or in combination means a straight-chain or branched hydrocarbon moiety comprising at least one carbon-carbon triple bond having 2 to 10, 2 to 20 or 2 to 50 carbon atoms, respectively. Each hydrogen atom of a C₂₋₁₀ alkynyl, C₂₋₂₀ alkynyl or C₂₋₅₀ alkynyl group may optionally be replaced by a substituent as defined below. Optionally, one or more double bond(s) may occur. Optionally, a C₂₋₁₀ alkynyl, C₂₋₂₀ alkynyl or C₂₋₅₀ alkynyl may be interrupted by one or more moieties as defined below.

As mentioned above, a C₁₋₄ alkyl, C₁₋₆ alkyl, C₁₋₁₀ alkyl, C₁₋₂₀ alkyl, C₁₋₅₀ alkyl, C₅₋₂₄ alkyl, C₂₋₆ alkenyl, C₂₋₁₀ alkenyl, C₂₋₂₀ alkenyl, C₂₋₅₀ alkenyl, C₂₋₆ alkynyl, C₂₋₁₀ alkynyl, C₂₋₂₀ alkenyl or C₂₋₅₀ alkynyl may optionally be interrupted by one or more moieties which in certain embodiments are selected from the group consisting of

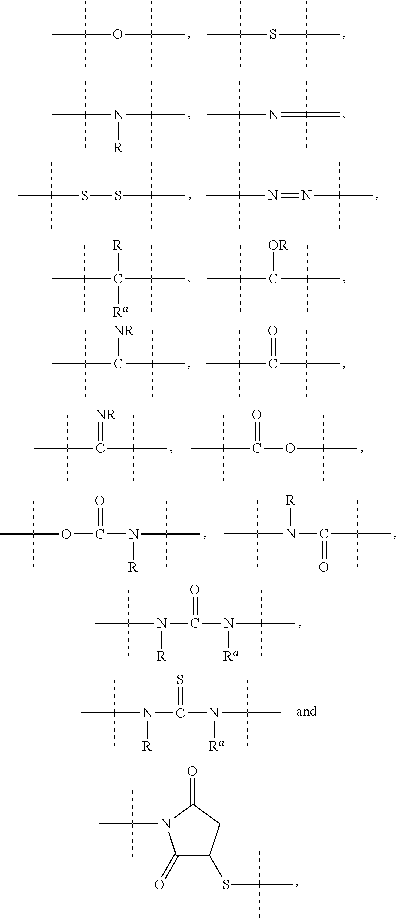

wherein
dashed lines indicate attachment to the remainder of the moiety or reagent;
—R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

As used herein, the term “C₃₋₁₀ cycloalkyl” means a cyclic alkyl chain having 3 to 10 carbon atoms, which may be saturated or unsaturated, e.g. cyclopropyl, cyclobutyl, cyclopentyl, cyclohexyl, cyclohexenyl, cycloheptyl, cyclooctyl, cyclononyl or cyclodecyl. Each hydrogen atom of a C₃₋₁₀ cycloalkyl carbon may be replaced by a substituent as defined below. The term “C₃₋₁₀ cycloalkyl” also includes bridged bicycles like norbornane or norbornene.

As used herein, the term “8- to 30-membered carbopolycyclyl” or “8- to 30-membered carbopolycycle” means a cyclic moiety of two or more rings with 8 to 30 ring atoms, where two neighboring rings share at least one ring atom and that may contain up to the maximum number of double bonds (aromatic or non-aromatic ring which is fully, partially or un-saturated). In certain embodiments, an 8- to 30-membered carbopolycyclyl means a cyclic moiety of two, three, four or five rings. In certain embodiments, an 8- to 30-membered carbopolycyclyl means a cyclic moiety of two, three or four rings.

As used herein, the term “3- to 10-membered heterocyclyl” or “3- to 10-membered heterocycle” means a ring with 3, 4, 5, 6, 7, 8, 9 or 10 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for 3- to 10-membered heterocycles include but are not limited to aziridine, oxirane, thiirane, azirine, oxirene, thiirene, azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine and homopiperazine. Each hydrogen atom of a 3- to 10-membered heterocyclyl or 3- to 10-membered heterocyclic group may be replaced by a substituent as defined below.

As used herein, the term “8- to 11-membered heterobicyclyl” or “8- to 11-membered heterobicycle” means a heterocyclic moiety of two rings with 8 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for an 8- to 11-membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine and pteridine. The term 8- to 11-membered heterobicycle also includes spiro structures of two rings like 1,4-dioxa-8-azaspiro[4.5]decane or bridged heterocycles like 8-aza-bicyclo[3.2.1]octane. Each hydrogen atom of an 8- to 11-membered heterobicyclyl or 8- to 11-membered heterobicycle carbon may be replaced by a substituent as defined below.

Similarly, the term “8- to 30-membered heteropolycyclyl” or “8- to 30-membered heteropolycycle” means a heterocyclic moiety of more than two rings with 8 to 30 ring atoms, in certain embodiments of three, four or five rings, where two neighboring rings share at least one ring atom and that may contain up to the maximum number of double bonds (aromatic or non-aromatic ring which is fully, partially or unsaturated), wherein at least one ring atom up to 10 ring atoms are replaced by a heteroatom selected from the group of sulfur (including —S(O)—, —S(O)₂—), oxygen and nitrogen (including ═N(O)—) and wherein the ring is linked to the rest of a molecule via a carbon or nitrogen atom.

As used herein, the term “excipient” refers to a diluent, adjuvant or vehicle with which the therapeutic, such as a drug or conjugate, is administered. Such pharmaceutical excipient can be sterile liquids, such as water and oils, including those of petroleum, animal, vegetable or synthetic origin, including but not limited to peanut oil, soybean oil, mineral oil, sesame oil and the like. Water is a preferred excipient when the pharmaceutical composition is administered orally. Saline and aqueous dextrose are preferred excipients when the pharmaceutical composition is administered intravenously. Saline solutions and aqueous dextrose and glycerol solutions are preferably employed as liquid excipients for injectable solutions. Suitable pharmaceutical excipients include starch, glucose, lactose, sucrose, mannitol, trehalose, gelatin, malt, rice, flour, chalk, silica gel, sodium stearate, glycerol monostearate, talc, sodium chloride, dried skim milk, glycerol, propylene, glycol, hyaluronic acid, propylene glycol, water, ethanol and the like. The pharmaceutical composition, if desired, can also contain minor amounts of wetting or emulsifying agents, pH buffering agents, like, for example, acetate, succinate, tris, carbonate, phosphate, HEPES (4-(2-hydroxyethyl)-1-piperazineethanesulfonic acid), MES (2-(N-morpholino)ethanesulfonic acid) or can contain detergents, like Tween®, poloxamers, poloxamines, CHAPS, Igepal® or amino acids like, for example, glycine, lysine or histidine. These pharmaceutical compositions can take the form of solutions, suspensions, emulsions, tablets, pills, capsules, powders, sustained-release formulations and the like. The pharmaceutical composition can be formulated as a suppository, with traditional binders and excipients such as triglycerides. Oral formulation can include standard excipients such as pharmaceutical grades of mannitol, lactose, starch, magnesium stearate, sodium saccharine, cellulose, magnesium carbonate, etc. Such compositions will contain a therapeutically effective amount of the drug or drug moiety, together with a suitable amount of excipient so as to provide the form for proper administration to the patient. The formulation should suit the mode of administration.

As used herein, the term “free form” of a drug refers to the drug in its unmodified, pharmacologically fully active form, e.g. after being released from the conjugate.

As used herein, the term “functional group” means a group of atoms which can react with other groups of atoms. Exemplary functional groups are carboxylic acid, primary amine, secondary amine, tertiary amine, maleimide, thiol, sulfonic acid, carbonate, carbamate, hydroxyl, aldehyde, ketone, hydrazine, isocyanate, isothiocyanate, phosphoric acid, phosphonic acid, haloacetyl, alkyl halide, acryloyl, aryl fluoride, hydroxylamine, disulfide, sulfonamides, sulfuric acid, vinyl sulfone, vinyl ketone, diazoalkane, oxirane and aziridine.

As used herein, the term “halogen” means fluoro, chloro, bromo or iodo. In certain embodiments, halogen is fluoro or chloro.

As used herein, the term “interrupted” means that a moiety is inserted in between two carbon atoms or—if the insertion is at one of the moiety's ends—between a carbon or heteroatom and a hydrogen atom, in certain embodiments between a carbon and a hydrogen atom.

In case the conjugates of the present invention comprise one or more acidic or basic groups, the invention also comprises their corresponding pharmaceutically or toxicologically acceptable salts, in particular their pharmaceutically utilizable salts. Thus, the conjugates of the present invention comprising acidic groups can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids, or quaternary ammoniums, such as tetrabutylammonium and cetyl trimethylammonium. Conjugates of the present invention comprising one or more basic groups, i.e. groups which can be protonated, can be present and can be used according to the invention in the form of their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, trifluoroacetic acid and other acids known to the person skilled in the art. For the person skilled in the art further methods are known for converting the basic group into a cation like the alkylation of an amine group resulting in a positively-charged ammonium group and an appropriate counterion of the salt. If the conjugates of the present invention simultaneously comprise acidic and basic groups, the invention also includes, in addition to the salt forms mentioned, inner salts or betaines (zwitterions). The respective salts can be obtained by customary methods, which are known to the person skilled in the art like, for example by contacting these prodrugs with an organic or inorganic acid or base in a solvent or dispersant, or by anion exchange or cation exchange with other salts. The present invention also includes all salts of the conjugates of the present invention which, owing to low physiological compatibility, are not directly suitable for use in pharmaceuticals but which can be used, for example, as intermediates for chemical reactions or for the preparation of pharmaceutically acceptable salts.

As used herein, the term “pharmaceutically acceptable” means a substance that does not cause harm when administered to a patient and preferably means approved by a regulatory agency, such as the EMA (Europe) and/or the FDA (US) and/or any other national regulatory agency for use in animals, preferably for use in humans.

As used herein, the term “peptide” as used herein refers to a chain of at least 2 and up to and including 50 amino acid monomer moieties, which may also be referred to as “amino acid residues”, linked by peptide (amide) linkages. The amino acid monomers may be selected from the group consisting of proteinogenic amino acids and non-proteinogenic amino acids and may be D- or L-amino acids. The term “peptide” also includes peptidomimetics, such as peptoids, beta-peptides, cyclic peptides and depsipeptides and covers such peptidomimetic chains with up to and including 50 monomer moieties.

As used herein, the term “protein” refers to a chain of more than 50 amino acid monomer moieties, which may also be referred to as “amino acid residues”, linked by peptide linkages, in which preferably no more than 12000 amino acid monomers are linked by peptide linkages, such as no more than 10000 amino acid monomer moieties, no more than 8000 amino acid monomer moieties, no more than 5000 amino acid monomer moieties or no more than 2000 amino acid monomer moieties.

As used herein, the term “small molecule drug” refers to drugs that are organic compounds with a molecular weight of less than 1000 Da, such as less than 900 Da or less than 800 Da. It is understood that nucleobase-based drug moieties, such as adenine or guanine analogues, may also be a type of small molecule drugs.

As used herein, the term “medium molecule drug” refers to drugs that are organic compounds which are not peptides and which are not proteins, and have a molecular weight ranging from and including 1 kDa to 7.5 kDa.

As used herein, the term “polymer” means a molecule comprising repeating structural units, i.e. the monomers, connected by chemical bonds in a linear, circular, branched, crosslinked or dendrimeric way or a combination thereof, which may be of synthetic or biological origin or a combination of both. The monomers may be identical, in which case the polymer is a homopolymer, or may be different, in which case the polymer is a heteropolymer. A heteropolymer may also be referred to as a “copolymer” and includes for example alternating copolymers in which monomers of different types alternate; periodic copolymers in which monomers of different types of monomers are arranged in a repeating sequence; statistical copolymers in which monomers of different types are arranged randomly; block copolymers in which blocks of different homopolymers consisting of only one type of monomers are linked by a covalent bond; and gradient copolymers in which the composition of different monomers changes gradually along a polymer chain. It is understood that a polymer may also comprise one or more other moieties, such as, for example, one or more functional groups. Likewise, it is understood that also a peptide or protein is a polymer, even though the side chains of individual amino acid residues may be different. It is understood that for covalently crosslinked polymers, such as hydrogels, no meaningful molecular weight ranges can be provided.

As used herein, the term “polymeric” or “polymeric moiety” refers to a reagent or a moiety comprising one or more polymers or polymer moieties. A polymeric reagent or moiety may optionally also comprise one or more other moiety/moieties, which in certain embodiments are selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl and tetralinyl;
branching points, such as —CR<, >C< or —N<; and
linkages selected from the group comprising

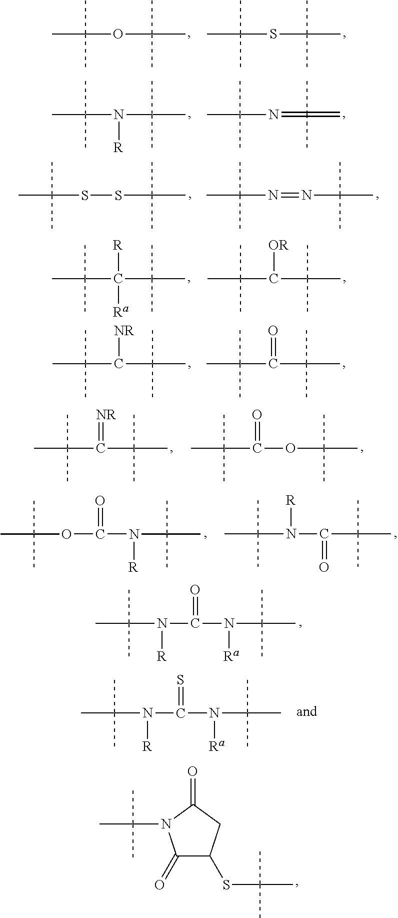

wherein
dashed lines indicate attachment to the remainder of the moiety or reagent;
—R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

The person skilled in the art understands that the polymerization products obtained from a polymerization reaction do not all have the same molecular weight, but rather exhibit a molecular weight distribution. Consequently, the molecular weight ranges, molecular weights, ranges of numbers of monomers in a polymer and numbers of monomers in a polymer as used herein, refer to the number average molecular weight and number average of monomers, i.e. to the arithmetic mean of the molecular weight of the polymer or polymeric moiety and the arithmetic mean of the number of monomers of the polymer or polymeric moiety.

Accordingly, in a polymeric moiety comprising “x” monomer units any integer given for “x” therefore corresponds to the arithmetic mean number of monomers. Any range of integers given for “x” provides the range of integers in which the arithmetic mean numbers of monomers lies. An integer for “x” given as “about x” means that the arithmetic mean numbers of monomers lies in a range of integers of x+/−10%, in certain embodiments lies in a range of integers x+/−8%, in certain embodiments lies in a range of integers x+/−5% and in certain embodiments lies in a range of integers x+/−2%.

As used herein, the term “number average molecular weight” means the ordinary arithmetic mean of the molecular weights of the individual polymers.

As used herein, the term “PEG-based” in relation to a moiety or reagent means that said moiety or reagent comprises PEG. In certain embodiments, such PEG-based moiety or reagent comprises at least 10% (w/w) PEG, such as at least 20% (w/w) PEG, such as at least 30% (w/w) PEG, such as at least 40% (w/w) PEG, such as at least 50% (w/w), such as at least 60% (w/w) PEG, such as at least 70% (w/w) PEG, such as at least 80% (w/w) PEG, such as at least 90% (w/w) PEG, such as at least 95% (w/w) PEG. The remaining weight percentage of the PEG-based moiety or reagent may be other moieties, such as those selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl and tetralinyl;
branching points, such as —CR<, >C< or —N<; and
linkages selected from the group comprising

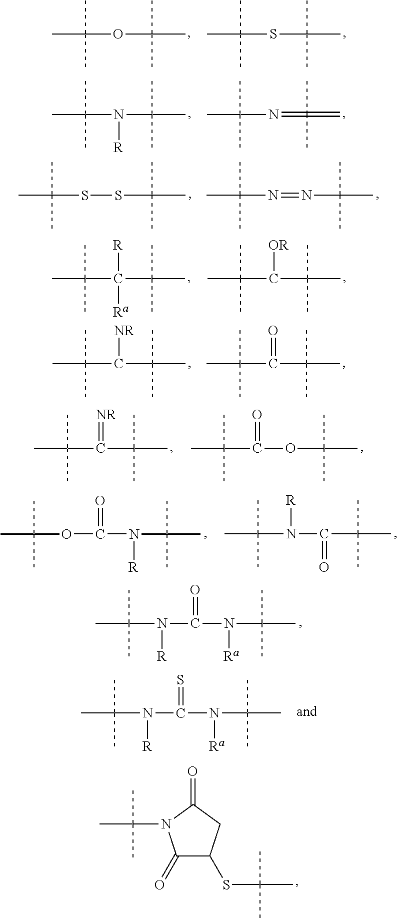

wherein
   dashed lines indicate attachment to the remainder of the moiety or reagent;
   —R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

As used herein, the term “PEG-based comprising at least X % PEG” in relation to a moiety or reagent means that said moiety or reagent comprises at least X % (w/w) ethylene glycol units (—CH₂CH₂O—), wherein the ethylene glycol units may be arranged blockwise, alternating or may be randomly distributed within the moiety or reagent. In certain embodiments, all ethylene glycol units of said moiety or reagent are present in one block; the remaining weight percentage of the PEG-based moiety or reagent are other moieties in certain embodiments selected from the group consisting of.
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl;
branching points, such as —CR<, >C< or —N<; and
linkages selected from the group comprising

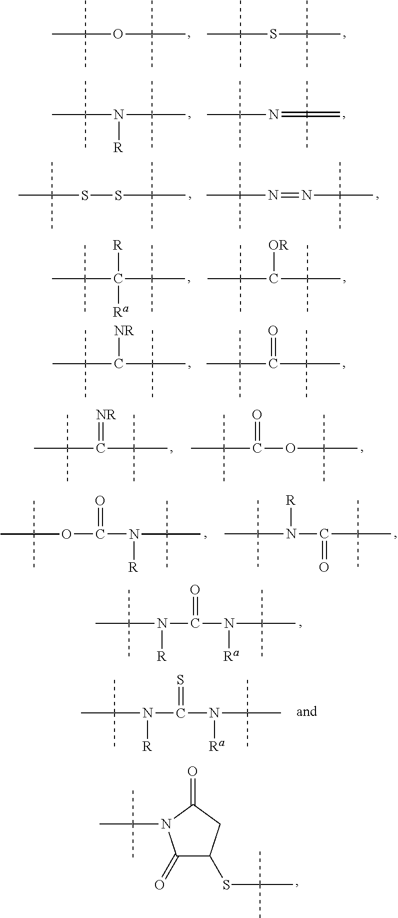

wherein
   dashed lines indicate attachment to the remainder of the moiety or reagent, and wherein —R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

As used herein, the term “hyaluronic acid-based” in relation to a moiety or reagent means that said moiety or reagent comprises hyaluronic acid. Such hyaluronic acid-based moiety or reagent comprises at least 10% (w/w) hyaluronic acid, such as at least 20% (w/w) hyaluronic acid, such as at least 30% (w/w) hyaluronic acid, such as at least 40% (w/w) hyaluronic acid, such as at least 50% (w/w) hyaluronic acid, such as at least 60% (w/w) hyaluronic acid, such as at least 70% (w/w) hyaluronic acid, such as at least 80% (w/w) hyaluronic acid, such as at least 90% (w/w) hyaluronic acid, or such as at least 95% (w/w) hyaluronic acid. The remaining weight percentage of the hyaluronic acid-based moiety or reagent may be other moieties, such as those selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl;
branching points, such as —CR<, >C< or —N<; and
linkages selected from the group consisting of

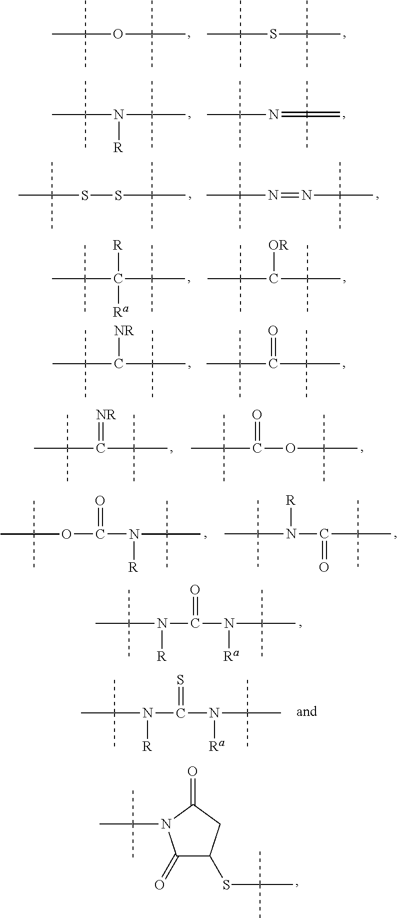

wherein
   dashed lines indicate attachment to the remainder of the moiety or reagent;
   —R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

As used herein, the term “hydrogel” means a hydrophilic or amphiphilic polymeric network composed of homopolymers or copolymers, which is insoluble due to the presence of hydrophobic interactions, hydrogen bonds, ionic interactions and/or covalent chemical crosslinks. The crosslinks provide the network structure and physical integrity.

As used herein, the term “random coil” refers to a peptide or protein adopting/having/forming, in certain embodiments having, a conformation which substantially lacks a defined secondary and tertiary structure as determined by circular dichroism spectroscopy performed in aqueous buffer at ambient temperature, and pH 7.4. In certain embodiments, the ambient temperature is about 20° C., i.e. between 18° C. and 22° C., while in certain embodiments the ambient temperature is 20° C.

As used herein, the term “spacer” or “spacer moiety” refers to a moiety suitable for connecting two moieties. Suitable spacers may be selected from the group consisting of C₁₋₅₀ alkyl, C₂₋₅₀ alkenyl and C₂₋₅₀ alkynyl, which C₁₋₅₀ alkyl, C₂₋₅₀ alkenyl or C₂₋₅₀ alkynyl is optionally interrupted by one or more groups selected from —NH—, —N(C₁₋₄ alkyl)-, —O—, —S—, —C(O)—, —C(O)NH—, —C(O)N(C₁₋₄ alkyl)-, —O—C(O)—, —S(O)—, —S(O)₂—, 4- to 7-membered heterocyclyl, phenyl and naphthyl and may optionally be substituted.

As used herein, the term “substituted” means that one or more —H atom(s) of a molecule or moiety are replaced by a different atom or a group of atoms, which are referred to as “substituent”.

As used herein, the term “substituent” refers in certain embodiments to a moiety selected from the group consisting of halogen, —CN, —C(O)OR^{x1}, —OR^{x1}, —C(O)R^{x1}, —C(O)N(R^{x1})(R^{x1a}), —S(O)₂N(R^{x1})(R^{x1a}), —S(O)N(R^{x1})(R^{x1a}), —S(O)₂R^{x1}, —S(O)R^{x1}, —N(R^{x1})S(O)₂N(R^{x1a})(R^{x1b}), —SR^{x1}, —N(R^{x1})(R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a})(R^{x1b}), —OC(O)N(R^{x1})(R^{x1a}), -T⁰, C¹⁻⁵⁰ alkyl, C₂₋₅₀ alkenyl and C₂₋₅₀ alkynyl; wherein -T⁰, C₁₋₅₀ alkyl, C₂₋₅₀ alkenyl and C₂₋₅₀ alkynyl are optionally substituted with one or more —R^{x2}, which are the same or different and wherein C₁₋₅₀ alkyl, C₂₋₅₀ alkenyl and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})_, —S—, —N(R^{x3})—, —OC(OR^{x3})(R^{x3a})—, —N(R^{x3})C(O)N(R^{x3a})— and —OC(O)N(R^{x3})—;

—R^{x1}, —R^{x1a}, —R^{x1b} are independently selected from the group consisting of —H, -T⁰, C₁₋₅₀ alkyl, C₂₋₅₀ alkenyl and C₂₋₅₀ alkynyl; wherein -T⁰, C₁₋₅₀ alkyl, C₂₋₅₀ alkenyl and C₂₋₅₀ alkynyl are optionally substituted with one or more —R^{x2}, which are the same or different and wherein C₁₋₅₀ alkyl, C₂₋₅₀ alkenyl and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})— and —OC(O)N(R^{x3})—;

each T⁰ is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T⁰ is independently optionally substituted with one or more —R^{x2}, which are the same or different;

each —R^{x2} is independently selected from the group consisting of halogen, —CN, oxo (═O), —C(O)OR^{x4}, —OR^{x4}, —C(O)R^{x4}, —C(O)N(R^{x4})(R^{x4a}), —S(O)₂N(R^{x4})(R^{x4a}), —S(O)N(R^{x4})(R^{x4a}), —S(O)₂R^{x4}, —S(O)R^{x4}, —N(R^{x4})S(O)₂N(R^{x4a})(R^{x4}b), —SR^{x4}, —N(R^{x4})(R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a})(R^{x4}b), —OC(O)N(R^{x4})(R^{x4a}) and C₁₋₆ alkyl; wherein C₁₋₆ alkyl is optionally substituted with one or more halogen, which are the same or different;

each —R^{x3}, —R^{x3a}, —R^{x4}, —R^{x4a}, —R^{x4b} is independently selected from the group consisting of —H and C₁₋₆ alkyl; wherein C₁₋₆ alkyl is optionally substituted with one or more halogen, which are the same or different.

In certain embodiments, the term “substituent” refers to a moiety selected from the group consisting of halogen, —CN, —COOR^{x1}, —OR^{x1}, —C(O)R^{x1}, —C(O)N(R^{x1})(R^{x1a}), —S(O)₂N(R^{x1})(R^{x1a}), —S(O)N(R^{x1})(R^{x1a}), —S(O)₂R^{x1}, —S(O)R^{x1}, —N(R^{x1})S(O)₂N(R^{x1})(R^{x1a}), —SR^{x1}, —N(R^{x1})(R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1})(R^{x1a}), —OC(O)N(R^{x1})(R^{x1a}), -T⁰, C₁₋₁₀ alkyl, C₂₋₁₀ alkenyl, and C₂₋₁₀ alkynyl; wherein -T⁰, C₁₋₁₀ alkyl, C₂₋₁₀ alkenyl, and C₂₋₁₀ alkynyl are optionally substituted with one or more —R^{x2}, which are the same or different and wherein C₁₋₁₀ alkyl, C₂₋₁₀ alkenyl, and C₂₋₁₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;

each —R^{x1}, —R^{x1a}, —R^{x1b}, —R^{x3}, —R^{x3a} is independently selected from the group consisting of —H, halogen, C₁₋₆ alkyl, C₂₋₆ alkenyl, and C₂₋₆ alkynyl;

each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2}, which are the same or different;

each —R^{x2} is independently selected from the group consisting of halogen, —CN, oxo (═O), —C(O)OR^{x4}, —OR^{x4}, —C(O)R^{x4}, —C(O)N(R^{x4})(R^{x4a}), —S(O)₂N(R^{x4})(R^{x4a}), —S(O)N(R^{x4})(R^{x4a}), —S(O)₂R^{x4}, —S(O)R^{x4}, —N(R^{x4})S(O)₂N(R^{x4a})(R^{x4}b), —SR^{x4}, —N(R^{x4})(R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a})(R^{x4b}), —OC(O)N(R^{x4})(R^{x4a}) and C₁₋₆ alkyl; wherein C₁₋₆ alkyl is optionally substituted with one or more halogen, which are the same or different;

each —R^{x4}, —R^{x4a}, —R^{x4b} is independently selected from the group consisting of —H, halogen, C₁₋₆ alkyl, C₂₋₆ alkenyl, and C₂₋₆ alkynyl.

In certain embodiments, the term “substituent” refers to a moiety selected from the group consisting of halogen, —CN, —COOR^{x1}, —OR^{x1}, —C(O)R^{x1}, —C(O)N(R^{x1})(R^{x1a}), —S(O)₂N(R^{x1})(R^{x1a}), —S(O)N(R^{x1})(R^{x1a}), —S(O)₂R^{x1}, —S(O)R^{x1}, —N(R^{x1})S(O)₂N(R^{x1a})(R^{x1b}), —SR^{x1}, —N(R^{x1})(R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a})(R^{x1b}), —OC(O)N(R^{x1})(R^{x1a}), -T⁰, C₁₋₆ alkyl, C₂₋₆ alkenyl, and C₂₋₆ alkynyl; wherein -T⁰, C₁₋₆ alkyl, C₂₋₆ alkenyl, and C₂₋₆ alkynyl are optionally substituted with one or more —R^{x2}, which are the same or different and wherein C₁₋₆ alkyl, C₂₋₆ alkenyl, and C₂₋₆ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;

each —R^{x1}, —R^{x1a}, —R^{x}b, —R^{x2}, —R^{x3}, —R^{x3a} is independently selected from the group consisting of —H, halogen, C₁₋₆ alkyl, C₂₋₆ alkenyl, and C₂₋₆ alkynyl;

each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2} which are the same or different.

In certain embodiments, a maximum of 6 —H atoms of an optionally substituted molecule are independently replaced by a substituent, e.g. 5 —H atoms are independently replaced by a substituent, 4 —H atoms are independently replaced by a substituent, 3 —H atoms are independently replaced by a substituent, 2 —H atoms are independently replaced by a substituent, or 1 —H atom is replaced by a substituent.

As used herein, the term “therapeutically effective amount” means an amount sufficient to cure, alleviate or partially arrest the clinical manifestations of a given disease and its complications. Effective amounts for each purpose will depend on the severity of the disease or injury as well as the weight and general state of the subject.

As used herein, the term “sustained-release” refers to the property of a compound, such as the conjugates of the present invention, to release a drug with a release half-life of at least 1 day.

As used herein, the term “water-insoluble” refers to a compound of which less than 1 g can be dissolved in one liter of water at 20° C. to form a homogeneous solution. Accordingly, the term “water-soluble” refers to a compound of which 1 g or more can be dissolved in one liter of water at 20° C. to form a homogeneous solution.

In general, the term “comprise(s)” or “comprising” also encompasses “consist of” or “consisting of”.

It is understood that the “N” in the phrase “π-electron-pair-donating heteroaromatic N” refers to nitrogen.

In certain embodiments, all moieties -D of the conjugate are identical, i.e. have the same chemical structure. In such case all moieties -D of the conjugate derive from the same type of drug molecule. It is understood that this means that all moieties -D originate from the same parent drug, but that there may be molecular rearrangements that for example lead to the formation of different tautomeric forms.

In certain embodiments, the conjugate of the present invention comprises different moieties -D, i.e. comprises moieties -D with different chemical structures. These different structures derive from different types of drug molecules. It is understood that this does not include certain molecular rearrangements that for example lead to the formation of different tautomeric forms, which however may also be present. In certain embodiments, the conjugate of the present invention comprises two different types of moieties -D. In certain embodiments, the conjugate of the present invention comprises three different types of moieties -D. In certain embodiments, the conjugate of the present invention comprises four different types of moieties -D. In certain embodiments, the conjugate of the present invention comprises five different types of moieties -D.

If the conjugates of the present invention comprise more than one type of -D, all moieties -D may be conjugated to the same type of -L¹- or may be conjugated to different types of -L¹-, i.e. a first type of -D may be conjugated to a first type of -L¹-, a second type of -D may be conjugated to a second type of -L¹- and so on. Using different types of -L¹- may, in certain embodiments, allow different release kinetics for different types of -D, such as for example a faster release for a first type of -D, a medium release for a second type of -D and a slow release for a third type of -D. Accordingly, in certain embodiments the conjugates of the present invention comprise one type of -L¹-. In certain embodiments, the conjugates of the present invention comprise two types of -L¹-. In certain embodiments, the conjugates of the present invention comprise three types of -L¹-. In certain embodiments, the conjugates of the present invention comprise four types of -L¹-.

In certain embodiments, the conjugates of the present invention comprise one type of -D and one type of -L¹-. In certain embodiments, the conjugates of the present invention comprise two types of -D and two types of -L¹-. In certain embodiments, the conjugates of the present invention comprise three types of -D and three types of -L¹-. In certain embodiments, the conjugates of the present invention comprise four types of -D and four types of -L¹-.

In certain embodiments, all moieties -L¹- of the conjugate have the same structure. In certain embodiments, the conjugate comprises two or more different types of moiety -L¹-, such as for example two, three, four or five different types of moiety -L¹-. Such two or more different types of moiety -L¹- may be conjugated to the same or different type of -D. Using different types of -L¹- allows releasing the same or different type of drug D-H from the conjugate of the present invention with different release half-lives, such as when combining a first group of moieties -L¹- with a short release half-life with a second group of moieties -L¹- with a long release half-life.

In certain embodiments, -D is selected from the group consisting of small molecule, medium size, peptide and protein drug moieties.

In certain embodiments, -D is a small molecule drug moiety. In certain embodiments, such small molecule drug moiety is a nucleobase-based drug moiety.

It is understood that a moiety -D may comprise at least one π-electron-pair-donating heteroaromatic nitrogen atoms, such as for example, one, two, three, four, five, six, seven, eight, nine or ten π-electron-pair-donating heteroaromatic nitrogen atoms. It is also understood that for the peptide and protein drug moieties such nitrogens may be provided by amino acids, such as for example, tryptophan or histidine and for nucleobase-based drug moieties such nitrogens may be provided by adenine or guanine.

In certain embodiments, -D is a peptide drug moiety.

In certain embodiments, -D is a peptide drug moiety selected from the group consisting of C-type natriuretic peptide, parathyroid hormone, W peptide, memno-peptide A and GI peptide.

In certain embodiments, -D is a protein drug moiety. In certain embodiments, such protein moiety is a monoclonal or polyclonal antibody or fragment or fusion thereof.

In certain embodiments, -D is selected from the group consisting of acitazanolast, seglitide, etodolac, ledazerol, N-desmethylmilameline, carbazomycin G, carbazomycin H, asperlicin C, asperlicin D, desacetylvinblastinehydrazide, jasplakinolide, ageliferin diacetate, ageliferin dihydrochloride, dolasetron, roxindole mesilate, liblomycin, tazanolast, abecamil, verticillatine, liarozole, irtemazole, omeprazole, parodilol hemifumarate, tropisetron, topsentine B1, bromotopsentin, lifarizine, pyrindamycin A, pyrindamycin B, duocarmycin C1, duocarmycin C2, duocarmycin A, biemnidin, elopiprazole, mibefradil, luzindole, manzamine D, manzamine B, manzamine C, octreotide acetate, lazabemide hydrochloride, tetrazolast meglumine, enalkiren, cloturin, pergolide mesylate, liarozole hydrochloride, chloropeptin II, adozelesin, carzelesin, beta-CCM, dexmedetomidine hydrochloride, naratriptan hydrochloride, indanomycin, homoindanomycin, mibefradil hydrochloride, dolasetron mesilate, nicotredole, duocarmycin B1, duocarmycin B2, vincristine sulfate, antiflammin-2, pantoprazole, manzamine A, janthinomycin C, albifylline, janthinomycin A, janthinomycin B, eflumast, voxergolide hydrochloride, gedocamil, temoporfin, proterguride, vinorelbine, cyclo[His-Pro], mepindolol transdermal patch, tubingensin B, methoxatin, mivazerol, atalaphillidine, atalaphillinine, discorhabdin D, lurosetron, naltrindole, azetirelin, bizelesin, intoplicine, cimetidine bismuth citrate, cimetidine bismuth L-tartrate, manzamine F, lecimibide, manzamine E, pyrazoloacridine, duocarmycin SA, vinfosiltine sulfate, nepaprazole, ramorelix, andolast, taltirelin, ramosetron hydrochloride, nafarelin acetate, cipamfylline, romergoline, pazelliptine trihydrochloride monohydrate, pazelliptine trihydrochloride, giracodazole, saviprazole, pibrozelesin hydrochloride, human angiotensin II, ceruletide diethylamine, carvedilol, remikiren mesilate, rolofylline, nortopsentin D, nortopsentin A, nortopsentin B, nortopsentin C, devazepide, atipamezole, imetit, batzelline B, carsatrin, demetomidine, medetomidine, pemetrexed disodium, carvotroline hydrochloride, sumatriptan succinate, alosetron maleate, leminoprazole, atevirdine mesylate, lifarizine hydrochloride, arofylline, nepaprazole, vinleucinol, moxonidine hydrochloride hydrate, lansoprazole, cytoblastin, L-histidinol, montirelin tetrahydrate, fabesetron hydrochloride, O6-benzylguanine, indisetron hydrochloride, pyrrolosporin A, antagonist-G, azatoxin, alpha-methyltryptophan, ecteinascidin 722, ecteinascidin 736, eptifibatide, dexpemedolac, kistamicin A, ilomastat, histrelin acetate, verongamine, spinorphin, delavirdine mesilate, epocarbazolin A, epocarbazolin B, ilatreotide, peldesine, prezatide copper acetate, plevitrexed, carquinostatin A, gavestinel sodium, thiazohalostatin, glycothiohexide alpha, cystamidin A, ciprokiren, immepip, immepyr, fipamezole hydrochloride, rizatriptan sulfate, clobenpropit, nomicotine, cabergoline, porfimer sodium, tezampanel, tenatoprazole, almotriptan, iodoproxyfan, pralmorelin, frovatriptan, pranazepide, rizatriptan benzoate, lomeguatrib, ¹¹¹In-pentetreotide, polydiscamide A, pimobendan, impentamine, apaxifylline, makaluvamine C, makaluvamine D, makaluvamine F, vinflunine, examorelin, pumosetrag hydrochloride, pranlukast hydrate, vilazodone hydrochloride, lanepitant, terguride, avitriptan, cimetidine, naxifylline, buserelin acetate, bopindolol, mepindolol sulfate, carprofen, leuprorelin acetate, oxypertine, elliptinium acetate, indoramin hydrochloride, reserpine, ergotamine tartrate, lisuride maleate, ilaprazole, chondramide A, chondramide B, chondramide C, chondramide D, lavanduquinocin, eletriptan, midaxifylline, indisulam, conivaptan hydrochloride, improgan, edotecarin, dexketoprofen imidazole salt, styloguanidine, ciproxifan, loloatin B, trifluproxim, nemifitide ditriflutate, beta-methyl-6-chloromelatonin, argyrin B, argyrin A, 18-hydroxycoronaridine, 18-methoxycoronaridine, fadolmidine hydrochloride, semaxanib, kurasoin B, avorelin, gilvusmycin, tegaserod maleate, carbazomadurin A, carbazomadurin B, rafabegron, nepadutant, donitriptan mesilate, becatecarin, donitriptan hydrochloride, Yttrium-90 edotreotide, methylhistaprodifen, histaprodifen, lemuteporfin, afeletecan hydrochloride, cipralisant, demethylasterriquinone B-1, indole-3-propionic acid, shermilamine D, decatromicin A, decatromicin B, venorphin, milbemycin alpha-9, alsterpaullone, secobatzelline B, arcyriacyanin A, O-demethylmurrayafoline A, clausenamine A, Secobatzelline A, sabiporide mesilate, alosetron hydrochloride, halimide, imoproxifan, barusiban, calothrixin A, golotimod, tadalafil, fluoroindolocarbazole C, fluoroindolocarbazole A, fluoroindolocarbazole B, denibulin hydrochloride, 99mTc-c(RGDfK*)2HYNIC, sunitinib, sunitinib malate, indolmycin, 2,7-dibromocryptolepine, pasireotide, calindol dihydrochloride, dacinostat, gilatide, pyridone-6, forodesine hydrochloride, sotrastaurin, gastrazole, yatakemycin, antileukinate, dovitinib lactate, axitinib, pruvanserin hydrochloride, shishijimicin C, shishijimicin A, shishijimicin B, plinabulin, DADMe-immucillin-G, DADMe-immucillin-H, cediranib, bremelanotide, immethridine, talaporfin sodium, methanobactin, [D-Tyr1] MS-10, [Arg(Me)9] MS-10, [D-Tyr1,Arg(Me)9] MS-10, [Trp19] MS-10, [D-Tyr1,AzaGly7,Arg(Me)9] MS-10, bederocin, methimepip, pachymedusa dacnicolor tryptophyllin-1, obatoclax mesylate, necrostatin-1, 2-bromo-7-nitrocryptolepine, 7-bromo-2-chlorocryptolepine, brivanib alaninate, brivanib, danusertib, afobazole, centanamycin, linifanib, ethylthio-DADMe-immucillin-A, 4-chlorophenylthio-DADMe-immucillin-A, methylthio-DADMe-immucillin-A, radezolid, shepherdin, desacetylvinblastinehydrazide folate conjugate, anamorelin hydrochloride, histrelin, mercaptopurine, histamine dihydrochloride, bleomycin A2 sulfate, bromocriptine mesilate, timodepressin, yohimbine, peplomycin, detomidine hydrochloride, vindesine, desglugastrin tromethamine, dihydroergotamine mesylate, oglufanide disodium, cefpimizole sodium, tinazoline hydrohloride, panobinostat, lanreotide acetate, pindolol, kinetin, reversine, carteramine A, meriolin-3, pymeprazole, 3-indole, PPI17-24, dexlansoprazole, lecirelin, methylhomoindanomycin, deslorelin, fabesetron, carmoxirole hydrochloride, galdansetron, melanotan II, nocathiacin II, theophylline, turofexorate isopropyl, marinopyrrole A, amycolamicin, calpinactam, microbisporicin A2, beta-amyloid (12-20), 5-fluorouracil, thioguanine, pemetrexed, mercaptopurine, tivantinib, ulixertinib, MK-8353, SCH772984, idelalisib, vemurafenib, EOS-200271 and X4P-001.

In certain embodiments, -D is axitinib.

In certain embodiments, —Y— is —N(R³)—. In certain embodiments, —Y— is —O—. In certain embodiments, —Y— is —S—.

In certain embodiments, —R¹, —R² and —R³ are independently selected from the group consisting of —H, -T, C₁₋₆ alkyl, C₂₋₆ alkenyl and C₂₋₆ alkynyl.

In certain embodiments, —R¹ is independently selected from the group consisting of —H, -T, C₁₋₆ alkyl, C₂₋₆ alkenyl and C₂₋₆ alkynyl. In certain embodiments, —R¹ is —H. In certain embodiments, —R¹ is -T. In certain embodiments, —R¹ is C₁₋₆ alkyl. In certain embodiments, —R¹ is C₂₋₆ alkenyl. In certain embodiments, —R¹ is C₂₋₆ alkynyl.

In certain embodiments, —R2 is independently selected from the group consisting of —H, -T, C₁₋₆ alkyl, C₂₋₆ alkenyl and C₂₋₆ alkynyl. In certain embodiments, —R2 is —H. In certain embodiments, —R² is -T. In certain embodiments, —R² is C₁₋₆ alkyl. In certain embodiments, —R² is C₂₋₆ alkenyl. In certain embodiments, —R² is C₂₋₆ alkynyl.

In certain embodiments, —R³ is independently selected from the group consisting of —H, -T, C₁₋₆ alkyl, C₂₋₆ alkenyl and C₂₋₆ alkynyl. In certain embodiments, —R³ is —H. In certain embodiments, —R³ is -T. In certain embodiments, —R³ is C₁₋₆ alkyl. In certain embodiments, —R³ is C₂₋₆ alkenyl. In certain embodiments, —R³ is C₂₋₆ alkynyl.

In certain embodiments, T is selected from the group consisting of phenyl, naphthyl, indenyl, indanyl, tetralinyl, C₃₋₁₀ cycloalkyl, 3- to 10-membered heterocyclyl and 8- to 11-heterobicyclyl. In certain embodiments, T is phenyl. In certain embodiments, T is naphthyl.

In certain embodiments, T is indenyl. In certain embodiments, T is indanyl. In certain embodiments, T is tetralinyl. In certain embodiments, T is C₃₋₁₀ cycloalkyl. In certain embodiments, T is 3- to 10-membered heterocyclyl. In certain embodiments, T is 8- to 11-heterobicyclyl.

In certain embodiments, T is substituted with one or more —R⁴. In certain embodiments, T is substituted with one —R⁴. In certain embodiments, T is not substituted with —R⁴.

In certain embodiments, —R⁴, —R⁵ and —R^{5a} are independently selected from the group consisting of —H and C₁₋₆ alkyl.

In certain embodiments, —R4 is selected from the group consisting of —H and C₁₋₆ alkyl. In certain embodiments, —R⁴ is —H. In certain embodiments, —R⁴ is C₁₋₆ alkyl.

In certain embodiments, —R⁵ is selected from the group consisting of —H and C₁₋₆ alkyl. In certain embodiments, —R⁵ is —H. In certain embodiments, —R⁵ is C₁₋₆ alkyl.

In certain embodiments, —R^{5a} is selected from the group consisting of —H and C₁₋₆ alkyl. In certain embodiments, —R^{5a} is —H. In certain embodiments, —R^{5a} is C₁₋₆ alkyl.

In certain embodiments, —Y— is —O— and —R² is C₁₋₆ alkyl. In certain embodiments, —Y— is —O— and —R² is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In certain embodiments, —Y— is —O— and —R² is methyl. In certain embodiments, —Y— is —O— and —R2 is ethyl.

In certain embodiments, —Y— is —O— and —R² is C₁₋₆ alkyl, wherein C₁₋₆ alkyl is interrupted by —C(O)—.

In certain embodiments, —Y— is —N(R³)— and —R² is C₁₋₆ alkyl, wherein C₁₋₆ alkyl is interrupted by —C(O)O— and —R³ is as defined in formula (I).

In certain embodiments, —Y— is —N(R³)— and —R² is C₁₋₆ alkyl, wherein C₁₋₆ alkyl is interrupted by —C(O)O— and —R³ is selected from the group consisting of —H, methyl, ethyl and propyl.

In certain embodiments, -L¹- of formula (I) is of formula (Ii):

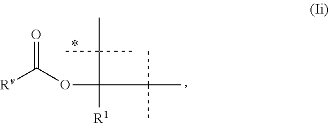

wherein
the dashed line marked with an asterisk indicates the attachment to -L²- and the unmarked dashed line indicates the attachment to the π-electron-pair-donating heteroaromatic N of -D;
—R^{v} is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and —R¹ is used as defined in formula (I).

In certain embodiments, —R^{v} of formula (Ii) is selected from the group consisting of methyl, ethyl and propyl. In certain embodiments, —R^{v} of formula (Ii) is methyl. In certain embodiments, —R^{v} of formula (Ii) is ethyl. In certain embodiments, —R^{v} of formula (Ii) is propyl.

In certain embodiments, -L¹- of formula (I) is of formula (Iii):

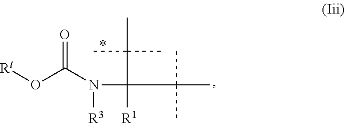

wherein
the dashed line marked with an asterisk indicates the attachment to -L²- and the unmarked dashed line indicates the attachment to the π-electron-pair-donating heteroaromatic N of -D;
R¹ is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and
—R¹ and —R³ are used as defined in formula (I).

In certain embodiments, —R³ of formula (Iii) is selected from the group consisting of —H, methyl and ethyl. In certain embodiments, —R³ of formula (Iii) is —H. In certain embodiments, —R³ of formula (Iii) is methyl. In certain embodiments, —R³ of formula (Iii) is ethyl.

In certain embodiments, —R^{t} of formula (Iii) is selected from the group consisting of methyl, ethyl and propyl. In certain embodiments, —R^{t} of formula (Iii) is methyl. In certain embodiments, —R^{t} of formula (Iii) is ethyl. In certain embodiments, —R^{t} of formula (Iii) is propyl.

In certain embodiments, -L¹- of formula (I) is of formula (Iiii):

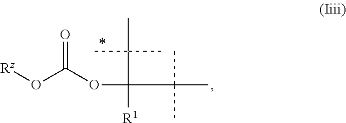

wherein
the dashed line marked with an asterisk indicates the attachment to -L²- and the unmarked dashed line indicates the attachment to the π-electron-pair-donating heteroaromatic N of -D;
R^{z} is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and —R¹ is used as defined in formula (I).

In certain embodiments, —R^{z} of formula (Iiii) is selected from the group consisting of methyl, ethyl and propyl. In certain embodiments, —R^{z} of formula (Iiii) is methyl. In certain embodiments, —R^{z} of formula (Iiii) is ethyl. In certain embodiments, —R^{z} of formula (Iiii) is propyl.

In certain embodiments, -L¹- is connected to -D through a heminal linkage. In certain embodiments, -L¹- is connected to -D through an aminal linkage. In certain embodiments, -L¹- is connected to -D through a hemithioaminal linkage. In certain embodiments, -L¹- is further substituted with one or more substituents.

In certain embodiments, -L¹- is not further substituted.

In certain embodiments, all moieties -L²- of the conjugate of the present invention are identical. In certain embodiments, the conjugate of the present invention comprises more than one type of -L²-, such as two, three, four or five different moieties -L²-. Such more than one type of -L²- may be connected to only one type of -L¹- or may be connected to more than one type of -L¹-.

In certain embodiments, -L²- is a chemical bond.

In certain embodiments, -L²- is a spacer moiety.

In certain embodiments, -L²- is selected from the group consisting of -T′-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl and C₂₋₅₀ alkynyl; wherein -T′-, C₁₋₅₀ alkyl, C₂₋₅₀ alkenyl and C₂₋₅₀ alkynyl are optionally substituted with one or more —R^{y2}, which are the same or different and wherein C₁₋₅₀ alkyl, C₂₋₅₀ alkenyl and C₂₋₅₀ alkynyl are optionally interrupted by one or more groups selected from the group consisting of -T′-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})— and —OC(O)N(R^{y3})—; —R^{y1} and —R^{y1a} are independently selected from the group consisting of —H, -T′, C₁₋₅₀ alkyl, C₂₋₅₀ alkenyl and C₂₋₅₀ alkynyl; wherein -T′, C₁₋₅₀ alkyl, C₂₋₅₀ alkenyl and C₂₋₅₀ alkynyl are optionally substituted with one or more —R^{y2}, which are the same or different, and wherein C₁₋₅₀ alkyl, C₂₋₅₀ alkenyl and C₂₋₅₀ alkynyl are optionally interrupted by one or more groups selected from the group consisting of -T′-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;

each T′ is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl; wherein each T′ is independently optionally substituted with one or more —R^{y2}, which are the same or different;

each —R^{y2} is independently selected from the group consisting of halogen, —CN, oxo (═O), —C(O)OR^{y5}, —OR^{y5}, —C(O)R^{y5}, —C(O)N(R^{y5})(R^{y5a}), —S(O)₂N(R^{y5})(R^{y5a}), —S(O)N(R^{y5})(R^{y5a}), —S(O)₂R^{y5}, —S(O)R^{y5}, —N(R^{y5})S(O)₂N(R^{y5})(R^{y5a}), —SR^{y5}, —N(R^{y5})(R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5})(R^{y5a}), —OC(O)N(R^{y5})(R^{y5a}), and C₁₋₆ alkyl; wherein C₁₋₆ alkyl is optionally substituted with one or more halogen, which are the same or different; and each —R^{y3}, —R^{y3a}, —R^{y4}, —R^{y4a}, —R^{y5}, —R^{y5a} and —R^{y5b} is independently selected from the group consisting of —H and C₁₋₆ alkyl; wherein C₁₋₆ alkyl is optionally substituted with one or more halogen, which are the same or different.

In certain embodiments, -L²- is selected from the group consisting of -T′-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T′-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T′-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R_{y3a})—, —S—, —N(R^{y3})—, —OC(ORY³)(R^{y3a})—, —N(R^{y3})C(O)N(R^{y3a})—, and —OC(O)N(R^{y3})—;

—R^{y1} and —R^{y1a} are independently selected from the group consisting of —H, -T′, C₁₋₁₀ alkyl, C₂₋₁₀ alkenyl, and C₂₋₁₀ alkynyl; wherein -T′, C₁₋₁₀ alkyl, C₂₋₁₀ alkenyl, and C₂₋₁₀ alkynyl are optionally substituted with one or more —R^{y2}, which are the same or different, and wherein C₁₋₁₀ alkyl, C₂₋₁₀ alkenyl, and C₂₋₁₀ alkynyl are optionally interrupted by one or more groups selected from the group consisting of -T′-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;

each T′ is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8 to 30-membered carbopolycyclyl, and 8- to 30-membered heteropolycyclyl; wherein each T′ is independently optionally substituted with one or more —R^{y2}, which are the same or different;

—R^{y2} is selected from the group consisting of halogen, —CN, oxo (═O), —C(O)OR^{y5}, —OR^{y5}, —C(O)R^{y5}, —C(O)N(R^{y5})(R^{y5a}), —S(O)₂N(R^{y5})(R^{y5a}), —S(O)N(R^{y5})(R^{y5a}), —S(O)₂R^{y5}, —S(O)R^{y5}, —N(R^{y5})S(O)₂N(R^{y5a})(R^{y5b}), —SR^{y5}, —N(R^{y5})(R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a})(R^{y5b}), —OC(O)N(R^{y5})(R^{y5a}) and C₁₋₆ alkyl; wherein C₁₋₆ alkyl is optionally substituted with one or more halogen, which are the same or different; and

each —R^{y3}, —R^{y3a}, —R^{y4}, —R^{y4a}, —R^{y5}, —R^{y5a} and —R^{y5b} is independently selected from the group consisting of —H and C₁₋₆ alkyl; wherein C₁₋₆ alkyl is optionally substituted with one or more halogen, which are the same or different.

In certain embodiments, -L²- is selected from the group consisting of -T′-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T′-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T′-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R_{y3})C(O)N(R^{y3a}), and —OC(O)N(R^{y3})—;

—R^{y1} and —R^{y1a} are independently selected from the group consisting of —H, -T′, C₁₋₁₀ alkyl, C₂₋₁₀ alkenyl and C₂₋₁₀ alkynyl;
each T′ is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8 to 30-membered carbopolycyclyl and 8- to 30-membered heteropolycyclyl;

each —R^{y2} is independently selected from the group consisting of halogen, and C₁₋₆ alkyl; and each —R^{y3}, —R^{y3a}, —R^{y4}, —R^{y4a}, —R^{y5}, —R^{y5a} and —R^{y5b} is independently selected from the group consisting of —H and C₁₋₆ alkyl; wherein C₁₋₆ alkyl is optionally substituted with one or more halogen, which are the same or different.

In certain embodiments, -L²- is a C₁₋₂₀ alkyl chain, which is optionally interrupted by one or more groups independently selected from the group consisting of —O—, -T′- and —C(O)N(R^{y1})—; and which C₁-20 alkyl chain is optionally substituted with one or more groups independently selected from the group consisting of —OH, -T′ and —C(O)N(R^{y6}R^{y6a}); wherein —R^{y1}, —R^{y6}, —R^{y6a} are independently selected from the group consisting of H and C₁₋₄ alkyl and wherein T′ is selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl.

In certain embodiments, -L²- has a molecular weight in the range of from 14 g/mol to 750 g/mol.

In certain embodiments, -L²- comprises a moiety selected from the group consisting of:

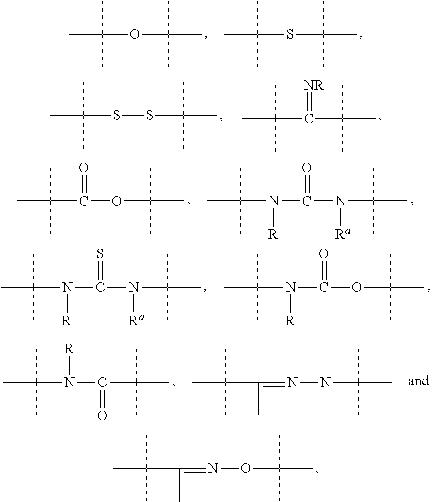

wherein dashed lines indicate attachment to -L¹-, the remainder of -L²- or Z, respectively; and —R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

In certain embodiments, Z is a polymeric moiety.

In certain embodiments, Z is a C₈₋₂₄ alkyl.

In certain embodiments, Z is water-soluble.

In certain embodiments, Z is a water-soluble polymeric moiety.

If Z is a water-soluble polymeric moiety, such polymeric moiety has a molecular weight ranging from and including 1 kDa to 1000 kDa. In certain embodiments, Z has a molecular weight ranging from and including 5 kDa to 1000 kDa. In certain embodiments, Z has a molecular weight ranging from and including 5 kDa to 500 kDa. In certain embodiments, Z has a molecular weight ranging from and including 10 kDa to 250 kDa. In certain embodiments, Z has a molecular weight ranging from and including 10 kDa to 150 kDa. In certain embodiments, Z has a molecular weight ranging from and including 12 kDa to 100 kDa. In certain embodiments, Z has a molecular weight ranging from and including 15 kDa to 80 kDa. In certain embodiments, Z has a molecular weight ranging from and including 10 kDa to 80 kDa.

In certain embodiments, Z has a molecular weight of about 80 kDa. In certain embodiments, Z has a molecular weight of about 70 kDa. In certain embodiments, Z has a molecular weight of about 60 kDa. In certain embodiments, Z has a molecular weight of about 50 kDa. In certain embodiments, Z has a molecular weight of about 40 kDa. In certain embodiments, Z has a molecular weight of about 30 kDa. In certain embodiments, Z has a molecular weight of about 20 kDa. In certain embodiments, Z has a molecular weight of about 10 kDa. In certain embodiments, Z has a molecular weight of about 5 kDa.

In certain embodiments, Z is a water-soluble polymeric moiety comprising a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

In certain embodiments, Z is a water-soluble polymeric moiety comprising a protein, such as a protein selected from the group consisting of carboxyl-terminal peptide of the chorionic gonadotropin as described in US 2012/0035101 A1 which are herewith incorporated by reference; albumin; XTEN sequences as described in WO 2011123813 A2 which are herewith incorporated by reference; proline/alanine random coil sequences as described in WO 2011/144756 A1 which are herewith incorporated by reference; proline/alanine/serine random coil sequences as described in WO 2008/155134 A1 and WO 2013/024049 A1 which are herewith incorporated by reference; and Fc-fusion proteins.

In certain embodiments, Z is a polysarcosine. In certain embodiments, Z comprises poly(N-methylglycine). In certain embodiments, Z comprises a random coil protein moiety. In certain embodiments, such random coil protein moiety comprises at least 25 amino acid residues and at most 2000 amino acids. In certain embodiments, such random coil protein moiety comprises at least 30 amino acid residues and at most 1500 amino acid residues. In certain embodiments, such random coil protein moiety comprises at least 50 amino acid residues and at most 500 amino acid residues.

In certain embodiments, Z comprises a random coil protein moiety of which at least 80%, in certain embodiments at least 85%, in certain embodiments at least 90%, in certain embodiments at least 95%, in certain embodiments at least 98% and in certain embodiments at least 99% of the total number of amino acids forming said random coil protein moiety are selected from alanine and proline. In certain embodiments, at least 10%, but less than 75%, in certain embodiments less than 65% of the total number of amino acid residues of such random coil protein moiety are proline residues. In certain embodiments, such random coil protein moiety is as described in WO 2011/144756 A1, which is hereby incorporated by reference in its entirety. In certain embodiments, Z comprises at least one moiety selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:51 and SEQ ID NO:61 as disclosed in WO2011/144756 which are hereby incorporated by reference. A moiety comprising such random coil protein comprising alanine and proline will be referred to as “PA” or “PA moiety”. Accordingly, in certain embodiments, Z comprises a PA moiety.

In certain embodiments, Z comprises a random coil protein moiety of which at least 80%, in certain embodiments at least 85%, in certain embodiments at least 90%, in certain embodiments at least 95%, in certain embodiments at least 98% and in certain embodiments at least 99% of the total number of amino acids forming said random coil protein moiety are selected from alanine, serine and proline. In certain embodiments, at least 4%, but less than 40% of the total number of amino acid residues of such random coil protein moiety are proline residues. In certain embodiments, such random coil protein moiety is as described in WO 2008/155134 A1, which is hereby incorporated by reference in its entirety. In certain embodiments, Z comprises at least one moiety selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:40, SEQ ID NO:42, SEQ ID NO:44, SEQ ID NO:46, SEQ ID NO:50, SEQ ID NO:52, SEQ ID NO:54 and SEQ ID NO:56 as disclosed in WO 2008/155134 A1, which are hereby incorporated by reference. A moiety comprising such random coil protein moiety comprising alanine, serine and proline will be referred to as “PAS” or “PAS moiety”. Accordingly, in certain embodiments, Z comprises a PAS moiety.

In certain embodiments, Z comprises a random coil protein moiety of which at least 80%, in certain embodiments at least 85%, in certain embodiments at least 90%, in certain embodiments at least 95%, in certain embodiments at least 98% and in certain embodiments 99% of the total number of amino acids forming said random coil protein moiety are selected from alanine, glycine, serine, threonine, glutamate and proline. In certain embodiments, such random coil protein moiety is as described in WO 2010/091122 A1 which is hereby incorporated by reference. In certain embodiments, Z comprises at least one moiety selected from the group consisting of SEQ ID NO:182, SEQ ID NO:183, SEQ ID NO:184; SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:759, SEQ ID NO:760, SEQ ID NO:761, SEQ ID NO:762, SEQ ID NO:763, SEQ ID NO:764, SEQ ID NO:765, SEQ ID NO:766, SEQ ID NO:767, SEQ ID NO:768, SEQ ID NO:769, SEQ ID NO:770, SEQ ID NO:771, SEQ ID NO:772, SEQ ID NO:773, SEQ ID NO:774, SEQ ID NO:775, SEQ ID NO:776, SEQ ID NO:777, SEQ ID NO:778, SEQ ID NO:779, SEQ ID NO:1715, SEQ ID NO:1716, SEQ ID NO:1718, SEQ ID NO:1719, SEQ ID NO:1720, SEQ ID NO:1721 and SEQ ID NO:1722 as disclosed in WO2010/091122A1, which are hereby incorporated by reference. A moiety comprising such random coil protein moiety comprising alanine, glycine, serine, threonine, glutamate and proline will be referred to as “XTEN” or “XTEN moiety” in line with its designation in WO 2010/091122 A1. Accordingly, in certain embodiments, Z comprises an XTEN moiety.

In certain embodiments, Z is a hyaluronic acid-based polymer.

In certain embodiments, Z is a polymeric moiety as disclosed in WO 2013/024047 A1 which is herewith incorporated by reference. In certain embodiments, Z is a polymeric moiety as disclosed in WO 2013/024048 A1 which is herewith incorporated by reference.

In certain embodiments, Z is a PEG-based polymer, such as linear, branched or multi-arm PEG-based polymer.

In certain embodiments, Z is a linear PEG-based polymer.

In certain embodiments, Z is a branched C₈₋₂₄ alkyl having one, two, three, four, five or six branching points. In certain embodiments, Z is a branched C₈₋₂₄ alkyl having one, two or three branching points. In certain embodiments, Z is a branched C₈₋₂₄ alkyl having one branching point. In certain embodiments, Z is a branched C₈₋₂₄ alkyl having two branching points. In certain embodiments, Z is a branched C₈₋₂₄ alkyl having three branching points.

In certain embodiments, Z is a branched polymer. In certain embodiments, Z is a branched polymer having one, two, three, four, five or six branching points. In certain embodiments, Z is a branched polymer having one, two or three branching points. In certain embodiments, Z is a branched polymer having one branching point. In certain embodiments, Z is a branched polymer having two branching points. In certain embodiments, Z is a branched polymer having three branching points.

In certain embodiments, a branching point is selected from the group consisting of —N<, —CH< and >C<. In certain embodiments, such branched moiety Z is PEG-based.

In certain embodiments, Z is a multi-arm PEG-based polymer. In certain embodiments, Z is a multi-arm PEG-based polymer having at least 2 PEG-based arms, such as 2, 3, 4, 5, 6, 7, or 8 PEG-based arms.

In certain embodiments, Z is a branched PEG-based polymer comprising at least 10% PEG, has one branching point and two PEG-based polymer arms and has a molecular weight of about 40 kDa. Accordingly, each of the two PEG-based polymer arms has a molecular weight of about 20 kDa. In certain embodiments, the branching point is —CH<.

In certain embodiments, Z is a branched PEG-based polymer comprising at least 10% PEG, has three branching points and four PEG-based polymer arms and has a molecular weight of about 40 kDa. Accordingly, each of the four PEG-based polymer arms has a molecular weight of about 10 kDa. In certain embodiments, each of the three branching points is —CH<.

In certain embodiments, Z is water-insoluble.

In certain embodiments, Z is a water-insoluble polymeric moiety.

In certain embodiments, Z is a water-insoluble polymeric moiety comprising a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

In certain embodiments, Z is a hydrogel.

In certain embodiments, Z is a PEG-based or hyaluronic acid-based hydrogel. In certain embodiments, Z is a PEG-based hydrogel. In certain embodiments, Z is a hyaluronic acid-based hydrogel.

In certain embodiments, Z is a hydrogel as described in WO 2006/003014 A2, WO 2011/012715 A1 or WO 2014/056926 A1, which are herewith incorporated by reference in their entirety.

In certain embodiments, Z is a polymer network formed through the physical aggregation of polymer chains, which physical aggregation is preferably caused by hydrogen bonds, crystallization, helix formation or complexation. In certain embodiments, such polymer network is a thermogelling polymer.

In certain embodiments, Z comprises a moiety selected from the group consisting of:

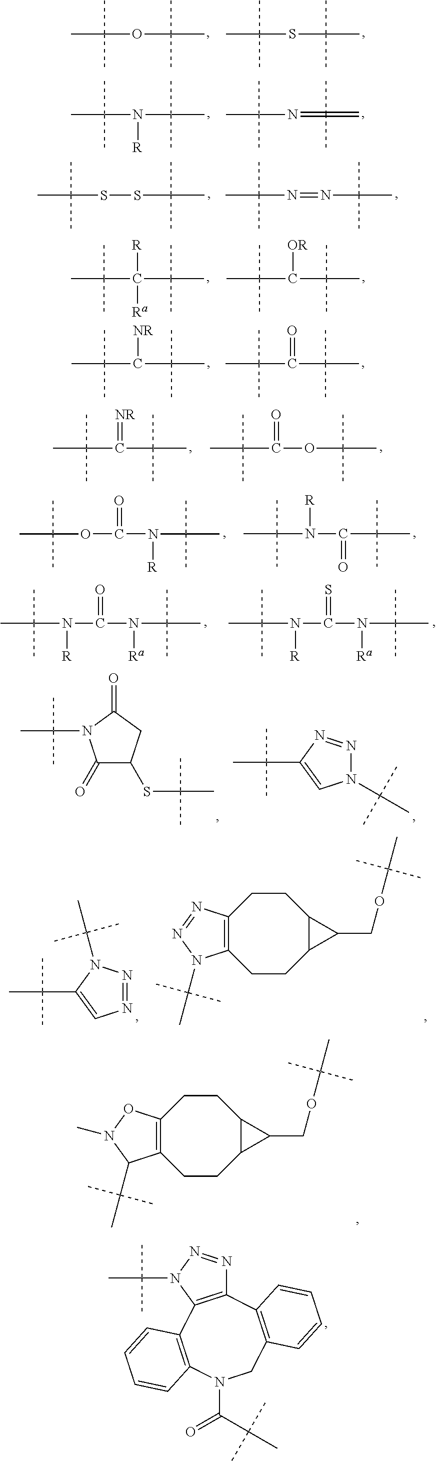

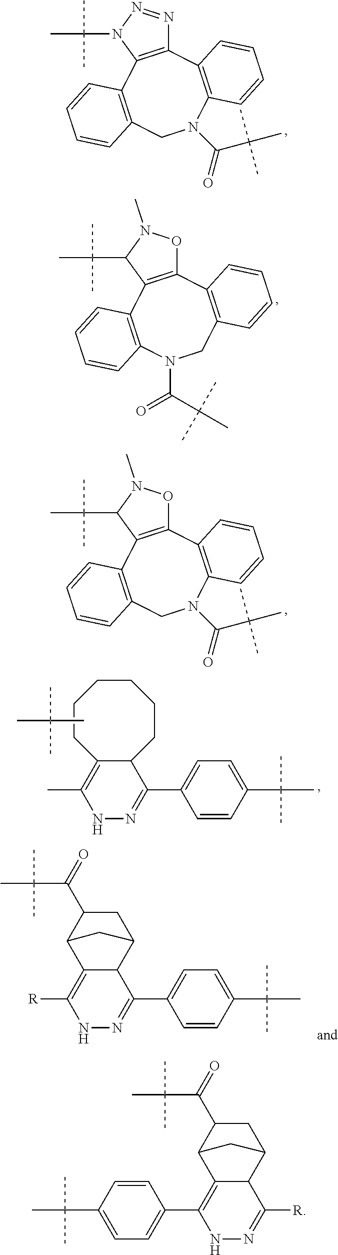

In certain embodiments, the conjugate of the present invention or the pharmaceutically acceptable salt thereof is of formula (Ia), (Ib), (Ic) or (Id):

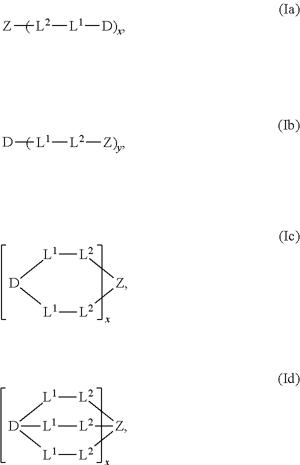

wherein
each -D, -L²- and Z are defined as above and each -L¹- is independently of formula (I);
x is an integer of at least 1; and
y is an integer selected from the group consisting of 2, 3, 4 and 5.

It is understood that even though one -D can be conjugated to multiple -L¹- moieties, the drug moiety is represented by “-D” and the drug by “D-H”.

In certain embodiments, the conjugate is of formula (Ia), (Ic) or (Id) and Z is a hydrogel. In such cases, a plurality of moieties -L²-L¹-D are conjugated to Z and it is understood that no upper limit for x can be provided.

In certain embodiments, the conjugate is of formula (Ia). In certain embodiments, the conjugate is of formula (Ib). In certain embodiments, the conjugate is of formula (Ic). In certain embodiments, the conjugate is of formula (Id). In certain embodiments, the conjugate is of formula (Ia) and Z is a hydrogel.

In certain embodiments, the conjugate is of formula (Ia), (Ic) or (Id), Z is a water-soluble polymeric moiety and x ranges from 2 to 1000, such as from 2 to 1500, such as from 2 to 1000, such as from 2 to 500, such as from 2 to 250 or such as from 2 to 100. In certain embodiments, the conjugate is of formula (Ia), (Ic) or (Id), Z is a water-soluble polymeric moiety and x is 20.

In certain embodiments, the conjugate is of formula (Ia), (Ic) or (Id), Z is a water-soluble polymeric moiety and x is 19. In certain embodiments, the conjugate is of formula (Ia), (Ic) or (Id), Z is a water-soluble polymeric moiety and x is 18. In certain embodiments, the conjugate is of formula (Ia), (Ic) or (Id), Z is a water-soluble polymeric moiety and x is 17. In certain embodiments, the conjugate is of formula (Ia), (Ic) or (Id), Z is a water-soluble polymeric moiety and x is 16. In certain embodiments, the conjugate is of formula (Ia), (Ic) or (Id), Z is a water-soluble polymeric moiety and x is 15. In certain embodiments, the conjugate is of formula (Ia), (Ic) or (Id), Z is a water-soluble polymeric moiety and x is 14. In certain embodiments, the conjugate is of formula (Ia), (Ic) or (Id), Z is a water-soluble polymeric moiety and x is 13. In certain embodiments, the conjugate is of formula (Ia), (Ic) or (Id), Z is a water-soluble polymeric moiety and x is 12. In certain embodiments, the conjugate is of formula (Ia), (Ic) or (Id), Z is a water-soluble polymeric moiety and x is 11. In certain embodiments, the conjugate is of formula (Ia), (Ic) or (Id), Z is a water-soluble polymeric moiety and x is 10. In certain embodiments, the conjugate is of formula (Ia), (Ic) or (Id), Z is a water-soluble polymeric moiety and x is 9. In certain embodiments, the conjugate is of formula (Ia), (Ic) or (Id), Z is a water-soluble polymeric moiety and x is 8. In certain embodiments, the conjugate is of formula (Ia), (Ic) or (Id), Z is a water-soluble polymeric moiety and x is 7. In certain embodiments, the conjugate is of formula (Ia), (Ic) or (Id), Z is a water-soluble polymeric moiety and x is 6. In certain embodiments, the conjugate is of formula (Ia), (Ic) or (Id), Z is a water-soluble polymeric moiety and x is 5. In certain embodiments, the conjugate is of formula (Ia), (Ic) or (Id), Z is a water-soluble polymeric moiety and x is 4. In certain embodiments, the conjugate is of formula (Ia), (Ic) or (Id), Z is a water-soluble polymeric moiety and x is 3. In certain embodiments, the conjugate is of formula (Ia), (Ic) or (Id), Z is a water-soluble polymeric moiety and x is 2.

In certain embodiments, the conjugate is of formula (Ib), Z is a water-soluble polymeric moiety and y is 1. In certain embodiments, the conjugate is of formula (Ib), Z is a water-soluble polymeric moiety and y is 2. In certain embodiments, the conjugate is of formula (Ib), Z is a water-soluble polymeric moiety and y is 3. In certain embodiments, the conjugate is of formula (Ib), Z is a water-soluble polymeric moiety and y is 4. In certain embodiments, the conjugate is of formula (Ib), Z is a water-soluble polymeric moiety and y is 5.

The conjugates of the present invention release one or more types of drug over an extended period of time, i.e. they are sustained-release conjugates. In certain embodiments, the release occurs with a release half-life ranging between 1 day and 1 month. In certain embodiments, the release occurs with a release half-life ranging between 1 day and 20 days. In certain embodiments, the release occurs with a release half-life between 1 day and 15 days. In certain embodiments the release half-life may also range from 2 to 20 days, 4 to 15 days or 3 to 6 days.

Another aspect of the present invention is a pharmaceutical composition comprising at least one conjugate of the present invention or a pharmaceutical salt thereof.

In certain embodiments, the pharmaceutical composition comprises one conjugate of the present invention or a pharmaceutical salt thereof. In certain embodiments, the pharmaceutical composition comprises two conjugates of the present invention. In certain embodiments, the pharmaceutical composition comprises three conjugates of the present invention.

Such pharmaceutical composition may have a pH ranging from pH 3 to pH 8, such as ranging from pH 4 to pH 6 or ranging from pH 4 to pH 5. In certain embodiments, the pH of the pharmaceutical composition is about 4. In certain embodiments, the pH of the pharmaceutical composition is about 4.5. In certain embodiments, the pH of the pharmaceutical composition is about 5. In certain embodiments, the pH of the pharmaceutical composition is about 5.5.

In certain embodiments, the pH of the pharmaceutical composition is 4. In certain embodiments, the pH of the pharmaceutical composition is 4.5. In certain embodiments, the pH of the pharmaceutical composition is 5. In certain embodiments, the pH of the pharmaceutical composition is 5.5.

In certain embodiments, the pH of the pharmaceutical composition ranges from pH 7 to pH 8.

In certain embodiments, the pH of the pharmaceutical composition is about 7. In certain embodiments, the pH of the pharmaceutical composition is about 8. In certain embodiments, the pH of the pharmaceutical composition is about 7.4. In certain embodiments, the pH of the pharmaceutical composition is about 7.5.

In certain embodiments, the pH of the pharmaceutical composition is 7. In certain embodiments, the pH of the pharmaceutical composition is 8. In certain embodiments, the pH of the pharmaceutical composition is 7.4. In certain embodiments, the pH of the pharmaceutical composition is 7.5.

In certain embodiments, such pharmaceutical composition is a suspension formulation.

In certain embodiments such pharmaceutical is a dry composition. It is understood that such dry composition may be obtained by drying, such as lyophilizing, a suspension composition.

If the pharmaceutical composition is a parenteral composition, suitable excipients may be categorized as, for example, buffering agents, isotonicity modifiers, preservatives, stabilizers, anti-adsorption agents, oxidation protection agents, viscosifiers/viscosity enhancing agents, anti-agglomeration agents or other auxiliary agents. However, in some cases, one excipient may have dual or triple functions. Excipient may be selected from the group consisting of

(i) Buffering agents: physiologically tolerated buffers to maintain pH in a desired range, such as sodium phosphate, bicarbonate, succinate, histidine, citrate, acetate, sulphate, nitrate, chloride, or pyruvate; antacids such as Mg(OH)₂ or ZnCO₃ may be also used;

(ii) Isotonicity modifiers: to minimize pain that can result from cell damage due to osmotic pressure differences at the injection depot; glycerin and sodium chloride are examples; effective concentrations can be determined by osmometry using an assumed osmolality of 285-315 mOsmol/kg for serum;

(iii) Preservatives and/or antimicrobials: multidose parenteral formulations require the addition of preservatives at a sufficient concentration to minimize risk of patients becoming infected upon injection and corresponding regulatory requirements have been established; typical preservatives include m-cresol, phenol, methylparaben, ethylparaben, propylparaben, butylparaben, chlorobutanol, benzyl alcohol, phenylmercuric nitrate, thimerosol, sorbic acid, potassium sorbate, benzoic acid, chlorocresol and benzalkonium chloride;

(iv) Stabilizers: Stabilisation is achieved by strengthening of the protein-stabilising forces, by destabilisation of the denatured state, or by direct binding of excipients to the protein; stabilizers may be amino acids such as alanine, arginine, aspartic acid, glycine, histidine, lysine, proline, sugars such as glucose, sucrose, trehalose, polyols such as glycerol, mannitol, sorbitol, salts such as potassium phosphate, sodium sulphate, chelating agents such as EDTA, hexaphosphate, ligands such as divalent metal ions (zinc, calcium, etc.), other salts or organic molecules such as phenolic derivatives; in addition, oligomers or polymers such as cyclodextrins, dextran, dendrimers, PEG or PVP or protamine or HSA may be used;

(v) Anti-adsorption agents: Mainly ionic or non-ionic surfactants or other proteins or soluble polymers are used to coat or adsorb competitively to the inner surface of the formulation's container; e.g., poloxamer (Pluronic F-68), PEG dodecyl ether (Brij 35), polysorbate 20 and 80, dextran, polyethylene glycol, PEG-polyhistidine, BSA and HSA and gelatins; chosen concentration and type of excipient depends on the effect to be avoided but typically a monolayer of surfactant is formed at the interface just above the CMC value; (vi) Oxidation protection agents: antioxidants such as ascorbic acid, ectoine, methionine, glutathione, monothioglycerol, morin, polyethylenimine (PEI), propyl gallate, and vitamin E; chelating agents such as citric acid, EDTA, hexaphosphate, and thioglycolic acid may also be used;

(vii) Viscosifiers or viscosity enhancers: retard settling of the particles in the vial and syringe and are used in order to facilitate mixing and resuspension of the particles and to make the suspension easier to inject (i.e., low force on the syringe plunger); suitable viscosifiers or viscosity enhancers are, for example, carbomer viscosifiers like Carbopol 940, Carbopol Ultrez 10, cellulose derivatives like hydroxypropylmethylcellulose (hypromellose, HPMC) or diethylaminoethyl cellulose (DEAE or DEAE-C), colloidal magnesium silicate (Veegum) or sodium silicate, hydroxyapatite gel, tricalcium phosphate gel, xanthans, carrageenans like Satia gum UTC 30, aliphatic poly(hydroxy acids), such as poly(D,L- or L-lactic acid) (PLA) and poly(glycolic acid) (PGA) and their copolymers (PLGA), terpolymers of D,L-lactide, glycolide and caprolactone, poloxamers, hydrophilic poly(oxyethylene) blocks and hydrophobic poly(oxypropylene) blocks to make up a triblock of poly(oxyethylene)-poly(oxypropylene)-poly(oxyethylene) (e.g. Pluronic®), polyetherester copolymer, such as a polyethylene glycol terephthalate/polybutylene terephthalate copolymer, sucrose acetate isobutyrate (SAIB), dextran or derivatives thereof, combinations of dextrans and PEG, polydimethylsiloxane, collagen, chitosan, polyvinyl alcohol (PVA) and derivatives, polyalkylimides, poly (acrylamide-co-diallyldimethyl ammonium (DADMA)), polyvinylpyrrolidone (PVP), glycosaminoglycans (GAGs) such as dermatan sulfate, chondroitin sulfate, keratan sulfate, heparin, heparan sulfate, hyaluronan, ABA triblock or AB block copolymers composed of hydrophobic A-blocks, such as polylactide (PLA) or poly(lactide-co-glycolide) (PLGA), and hydrophilic B-blocks, such as polyethylene glycol (PEG) or polyvinyl pyrrolidone; such block copolymers as well as the abovementioned poloxamers may exhibit reverse thermal gelation behavior (fluid state at room temperature to facilitate administration and gel state above sol-gel transition temperature at body temperature after injection);

(viii) Spreading or diffusing agent: modifies the permeability of connective tissue through the hydrolysis of components of the extracellular matrix in the intrastitial space such as but not limited to hyaluronic acid, a polysaccharide found in the intercellular space of connective tissue; a spreading agent such as but not limited to hyaluronidase temporarily decreases the viscosity of the extracellular matrix and promotes diffusion of injected drugs;

(ix) Anti-agglomeration agents, such as propylene glycol; and

(x) Other auxiliary agents: such as wetting agents, viscosity modifiers, antibiotics, hyaluronidase; acids and bases such as hydrochloric acid and sodium hydroxide are auxiliary agents necessary for pH adjustment during manufacture.

In another aspect, the present invention relates to a conjugate of the present invention or a pharmaceutical composition comprising a conjugate of the present invention for use as a medicament.

In another aspect, the present invention relates to a conjugate or a pharmaceutically acceptable salt thereof of the present invention or a pharmaceutical composition comprising a conjugate of the present invention for use in a method of treating a disease that can be treated with D-H or its pharmaceutically acceptable salt thereof.

In a further aspect, the present invention relates to a method of preventing a disease or treating a patient suffering from a disease that can be prevented or treated with D-H comprising administering an effective amount of the conjugate or its pharmaceutically acceptable salt thereof of the present invention or the pharmaceutical compositions comprising said conjugates to the patient.

As the present invention is applicable to all drug molecules comprising a π-electron-pair-donating heteroaromatic N, it is impossible to further specify the disease that can be treated. However, it is evident to the person skilled in the art which disease can be treated with a particular conjugate.

### EXAMPLES

Materials and Methods

All materials were commercially available except where stated otherwise.

Reactions

Reactions were performed with dry solvents (CH₂Cl₂, DMF, THF) stored over molecular sieves purchased from Sigma-Aldrich Chemie GmbH, Munich, Germany. Generally, reactions were stirred at room temperature and monitored by LCMS.

Flash Chromatography

Flash chromatography purifications were performed on an Isolera One system or an Isolera Four system from Biotage AB, Sweden, using Biotage KP-Sil silica cartridges. Products were detected at 254 nm or 280 nm.

RP-HPLC Purification

Preparative RP-HPLC purifications were performed with a Waters 600 controller with a 2487 Dual Absorbance Detector or an Agilent Infinity 1260 preparative system using a Waters XBridge BEH300 Prep C18 10 μm, 150×30 mm column as stationary phase. Products were detected at 215 nm, 320 nm or 360 nm. Linear gradients of solvent system A (water containing 0.1% TFA v/v) and solvent system B (acetonitrile containing 0.1% TFA v/v) were used. HPLC fractions containing product were pooled and lyophilized if not stated otherwise.

UPLC-MS Analysis

Analytical ultra-performance LC (UPLC)-MS was performed on a Waters Acquity system or an Agilent 1290 Infinity II equipped with a Waters BEH300 C18 column (2.1×50 mm, 1.7 μm particle size or 2.1×100 mm, 1.7 μm particle size); solvent A: water containing 0.04% TFA (v/v), solvent B: acetonitrile containing 0.05% TFA (v/v) coupled to a Waters Micromass ZQ or coupled to an Agilent Single Quad MS system.

### Example 1: Synthesis of Compound 1

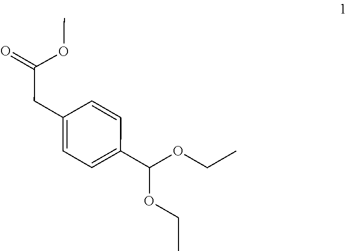 [SMILES: CCOC(OCC)c1ccc(CC(=O)OC)cc1]

The commercially available materials methyl 2-(4-formylphenyl)acetate (1.0 eq) and tripropyl orthoformate (3.0 eq) are combined in EtOH with a catalytic amount of TsOH (0.05 eq) and heated at reflux for 4 h. After removing the volatiles in vacuo, the material is purified by normal-phase flash chromatography.

In an alternative synthesis route for 1 tripropyl orthoformate is replaced by triethyl orthoformate.

### Example 2: Synthesis of Compound 2

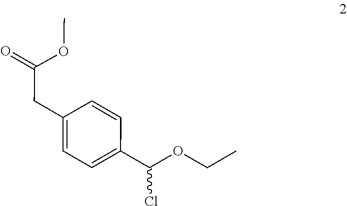 [SMILES: CCOC(Cl)c1ccc(CC(=O)OC)cc1]

Under N₂, a solution of 1 (1.0 eq) in anhydrous THF is cooled in an ice-bath before addition of thionyl chloride (0.2 eq) and acetic acid (1.5 eq). The solution is warmed to rt before being heated at reflux for 1 h. The volatiles are removed in vacuo.

### Example 3: Synthesis of Compound 3b

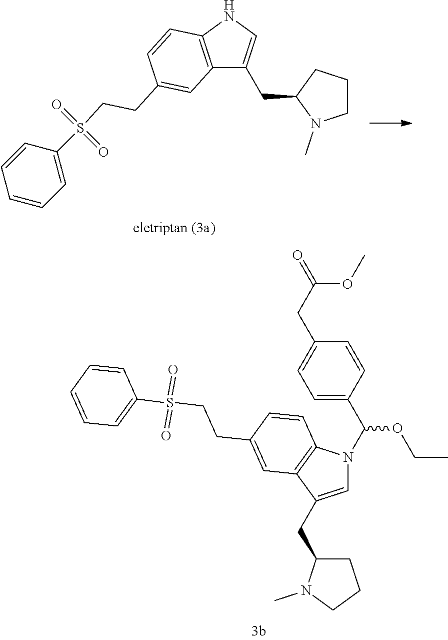 [SMILES: CN1CCC[C@@H]1Cc1c[nH]c2ccc(CCS(=O)(=O)c3ccccc3)cc12 ; CCOC(c1ccc(CC(=O)OC)cc1)n1cc(C[C@H]2CCCN2C)c2cc(CCS(=O)(=O)c3ccccc3)ccc21]

Under a N₂ atmosphere, eletriptan (3a) (1.0 eq) is dissolved in anhydrous DMF before addition of sodium hydride (1.2 eq). After stirring for 30 min, compound 2 (1.2 eq) is added and the mixture stirred at rt for 16 h. The solvent is removed in vacuo and the crude material purified by prep-HPLC.

### Example 4: Synthesis of Compound 4b

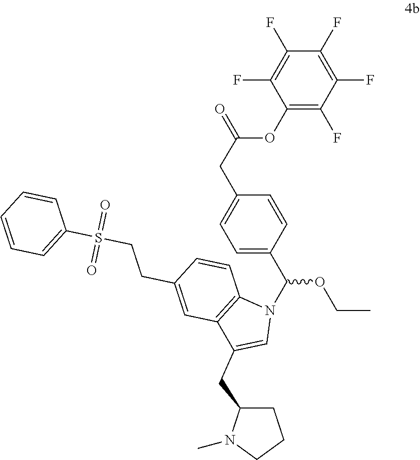 [SMILES: CCOC(c1ccc(CC(=O)Oc2c(F)c(F)c(F)c(F)c2F)cc1)n1cc(C[C@H]2CCCN2C)c2cc(CCS(=O)(=O)c3ccccc3)ccc21]

Compound 3b (1.0 eq) is stirred in 2 M aq. LiOH for 18 h, before acidification with 1 M aq. HCl. After 3× extraction into DCM, the combined organics are dried over MgSO₄ and the volatiles removed in vacuo to give 4a.

Compound 4a (1.0 eq) is combined with bis(pentafluorophenyl)carbonate (2.0 eq) in DMF. To the mixture is added DIPEA (6.0 eq) before stirring for 2 h. The solvent is removed in vacuo. Purification is performed via prep-HPLC and only non-buffered solvents are used and the pure fractions freeze-dried immediately upon collection to give 4b.

### Example 5: Synthesis of Compound 5b

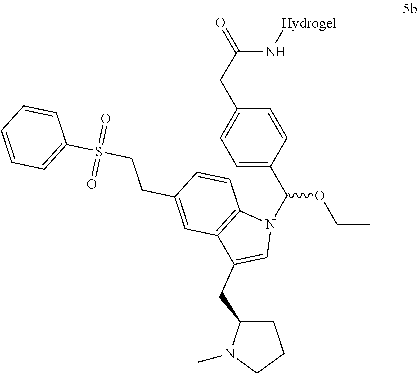

The amine-functionalized PEG-based hydrogel 5a is synthesized as described in WO 2011/012715 A1, Example 3. The hydrogel is swollen in 1% DIPEA in DMF in a syringe reactor with frit and washed three times with a 1% DIPEA/DMF solution. 4b (2.0 eq per hydrogel amine) is dissolved in 1% DIPEA in DMF. The solution is drawn into the hydrogel-containing reactor and shaken for 16 h at rt. The syringe is drained, the hydrogel washed several times with DMF, then washed several times with pH 5.5 20 mM sodium succinate aqueous buffer. A hydrogel suspension in pH 5.5 aqueous buffer is obtained.

### Example 6: Synthesis of Compound 6

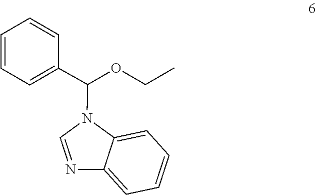 [SMILES: CCOC(c1ccccc1)n1cnc2ccccc21]

To benzaldehyde diethylacetal (51 μl, 0.28 mmol) was added acetyl chloride (100 μl, 1.4 mmol) and thionyl chloride (20 μl, 0.28 mmol) under an argon atmosphere and the mixture was heated to 65° C. for 90 min. The heating was stopped, and the mixture was concentrated under reduced pressure. Benzimidazole (32.8 g, 0.28 mmol) was dissolved in THF (250 μL) and DMF (200 μl), the mixture was cooled to 0° C., and n-butyl lithium (2.5 M in hexane, 122.1 μl, 0.31 mmol) was added dropwise. After complete addition the cooling bath was removed, and the mixture was stirred at ambient temperature for 30 min. The crude benzaldehyde diethylacetal product was dissolved in THF (250 μl) and added dropwise. After complete addition the reaction mixture was stirred at ambient temperature for 3 h 15 min. The reaction mixture was quenched with saturated Na₂CO₃ (1 ml) and diluted with H₂O (3 ml). The mixture was extracted with DCM (3×1 ml). The combined organic extracts were dried with Na₂SO₄, filtrated and concentrated under reduced pressure. 6 was purified using flash chromatography.

Yield: 16 mg (22.9%)

MS: m/z 253.20=[M+H]⁺, (calculated=253.13).

### Example 7: Synthesis of Compound 7

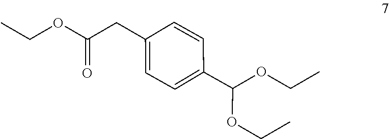 [SMILES: CCOC(=O)Cc1ccc(C(OCC)OCC)cc1]

A solution of ethyl 2-(4-formylphenyl)acetate (100 mg, 0.52 mmol) in triethyl orthoformate (500 μl) was cooled to 0° C. Amberlyst 15 (hydrogen form, 25 mg) was added, and the mixture was stirred at 0° C. for 5.5 h. The cooling bath was removed, and the reaction mixture was stirred for 18 h. The reaction mixture was diluted with ethyl acetate (20 ml) filtrated and concentrated under reduced pressure. 7 was purified using flash chromatography.

Yield: 110 mg (75.4%)

NMR: ¹H-NMR (43 MHz, CHCl₃-d₁): δ=1.24 (t, J=7.0 Hz), 3.34-4.40 (m), 7.34-7.38 (m).

### Example 8: Synthesis of Compound 8

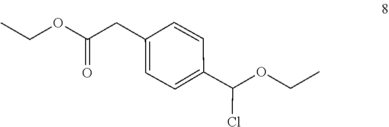 [SMILES: CCOC(=O)Cc1ccc(C(Cl)OCC)cc1]

Compound 7 (110 mg, 0.39 mmol) was dissolved in acetyl chloride (200 μl, 2.8 mmol) and thionyl chloride (40 μl, 0.55 mmol) under an argon atmosphere and the mixture was heated to 65° C. for 90 min. The mixture was concentrated under reduced pressure and the crude material was dried under high vacuum for 20 min. The obtained material was dissolved in THF (400 μl) and used directly in the next step.

### Example 9: Synthesis of Compound 9

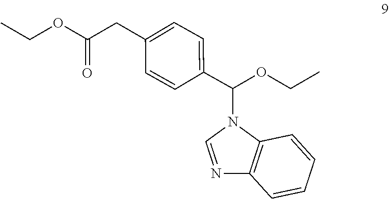 [SMILES: CCOC(=O)Cc1ccc(C(OCC)n2cnc3ccccc32)cc1]

A solution of benzimidazole (23.2 mg, 196.2 μmol) in THF (250 μl) and DMF (200 μl) was cooled to 0° C. and n-butyl lithium (2.5 M in hexane, 86.3 μl, 215.8 μmol) was added dropwise. After complete addition the cooling bath was removed, and the mixture was stirred at ambient temperature for 50 min. Compound 8 (50.4 mg, 196.2 μmol) was added dropwise as a solution in THF (200 μl). After complete addition the reaction mixture was stirred for 2 h 40 min. The reaction mixture was quenched by addition of saturated NaHCO₃ (1 ml) and extracted with DCM (3×1.5 ml). The combined organic layers were dried with Na₂SO₄, filtrated and concentrated under reduced pressure. 9 was purified using flash chromatography.

Yield: 11.9 mg (17.9%)

MS: m/z 339.13=[M+H]⁺, (calculated=339.17).

### Example 10: Synthesis of Compound 10

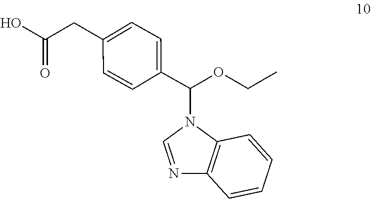 [SMILES: CCOC(c1ccc(CC(=O)O)cc1)n1cnc2ccccc21]

To a solution of compound 9 (5 mg, 13.9 μmol) in THF (200 μl) and water (100 μl) was added LiOH (1.75 mg, 41.7 μmol) and the mixture was stirred at ambient temperature for 1 h 20 min. 10 was purified using RP-HPLC purification without additional TFA.

Yield: 3.8 mg (88.2%) MS: m/z 311.16=[M+H]⁺, (calculated=311.14).

### Example 11: Synthesis of Compound 11

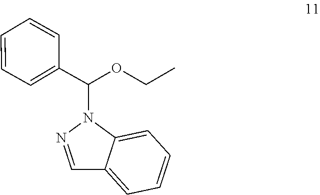 [SMILES: CCOC(c1ccccc1)n1ncc2ccccc21]

Compound 11 was prepared according to the synthesis procedure for compound 6.

Yield: 13 mg (18.6%)

MS: m/z 275.19=[M+Na]⁺, (calculated=275.12).

### Example 12: In Vitro Release Kinetics

The cleavage rate of the reversible bond from conjugates 6, 10 and 11 was monitored at pH 7.4 and 37° C. in aqueous buffer (pH 7.4 48 mM sodium phosphate, 20% acetonitrile). The disappearance of the conjugate was determined by LCMS (UV detection) and fitted with curve fitting software to obtain the half-life of the release.

| Compound | t_{1/2} (pH 7.4) | Released product |
|---|---|---|
| 6 | 8.0 d | 1H-benzo[d]imidazole |
| 10 | 10 h | 1H-benzo[d]imidazole |
| 11 | 5.7 d | indazole |

### Example 13: Synthesis of Compound 12b

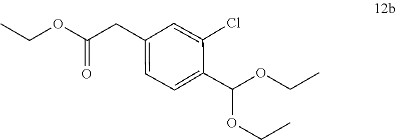 [SMILES: CCOC(=O)Cc1ccc(C(OCC)OCC)c(Cl)c1]

The commercially available material 3-chloro-4-formylbenzeneacetic acid (1.0 eq) is dissolved in anhydrous DCM and absolute EtOH. Thionyl chloride (2 eq) is added and the reaction mixture is refluxed for 16 h. The solution is cooled and concentrated in vacuo. The obtained crude material is dissolved in DCM and washed with saturated NaHCO₃. The organic layer is dried with Na₂SO₄ and concentrated in vacuo to give 12a.

To a solution of 12a (1.0 eq) in absolute EtOH is added triethyl orthoformate (3.5 eq) and a catalytic amount of concentrated HCl. The mixture is allowed to reflux for 18 h. The solution is cooled and concentrated in vacuo. The obtained crude material is taken up in diethyl ether, washed with 2M NaOH, the organic layer is dried with Na₂SO₄ and concentrated in vacuo. Compound 12b is purified by flash chromatography.

### Example 14: Synthesis of Compound 13

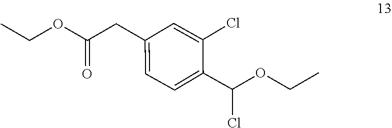 [SMILES: CCOC(=O)Cc1ccc(C(Cl)OCC)c(Cl)c1]

Compound 13 is prepared from compound 12b according to the synthesis procedure for example 8.

### Example 15: Synthesis of Compound 14

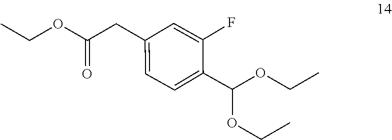 [SMILES: CCOC(=O)Cc1ccc(C(OCC)OCC)c(F)c1]

Starting from commercially available material 3-fluoro-4-formylbenzeneacetic acid, compound 14 is prepared according to the procedure for example 13.

### Example 16: Synthesis of Compound 15

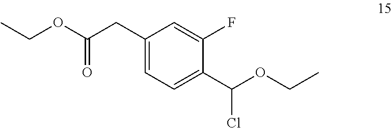 [SMILES: CCOC(=O)Cc1ccc(C(Cl)OCC)c(F)c1]

Compound 15 is prepared from compound 14 according to the synthesis procedure for example 8.

### Example 17: Synthesis of Compound 16

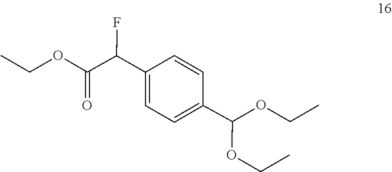 [SMILES: CCOC(=O)C(F)c1ccc(C(OCC)OCC)cc1]

Compound 16 is synthesized according to the procedure described by Wu et al. in Organic Letters 2016, 18, 5564-5567.

### Example 18: Synthesis of Compound 17

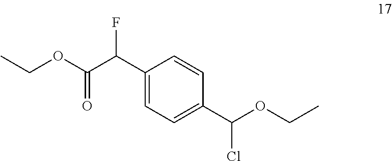 [SMILES: CCOC(=O)C(F)c1ccc(C(Cl)OCC)cc1]

Compound 17 is prepared from compound 16 according to the synthesis procedure for example 8.

### Example 19: Synthesis of Compound 18b

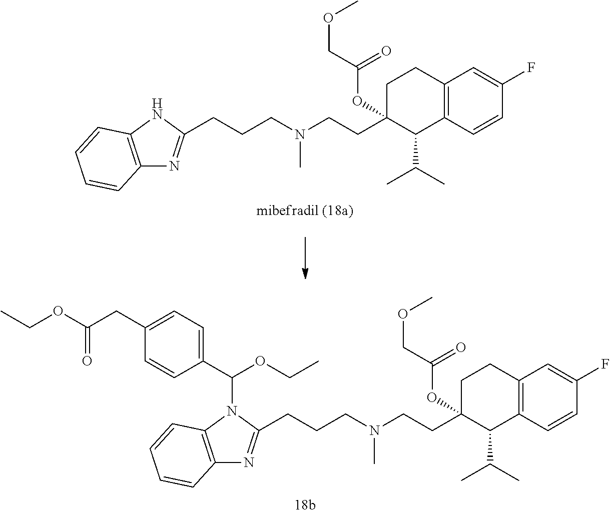 [SMILES: COCC(=O)O[C@]1(CCN(C)CCCc2nc3ccccc3[nH]2)CCc2cc(F)ccc2[C@@H]1C(C)C ; CCOC(=O)Cc1ccc(C(OCC)n2c(CCCN(C)CC[C@@]3(OC(=O)COC)CCc4cc(F)ccc4[C@@H]3C(C)C)nc3ccccc32)cc1]

Under a N₂ atmosphere, mibefradil (18a, 1 eq) is dissolved in anhydrous THF before addition of sodium hydride (1.2 eq). After stirring for 30 min, a solution of compound 8 (1.1 eq) in anhydrous THF is added, and the mixture is stirred for 16 h. The solvent is removed in vacuo and the crude material purified by flash chromatography to give 18b.

### Example 20: Synthesis of Compound 19b

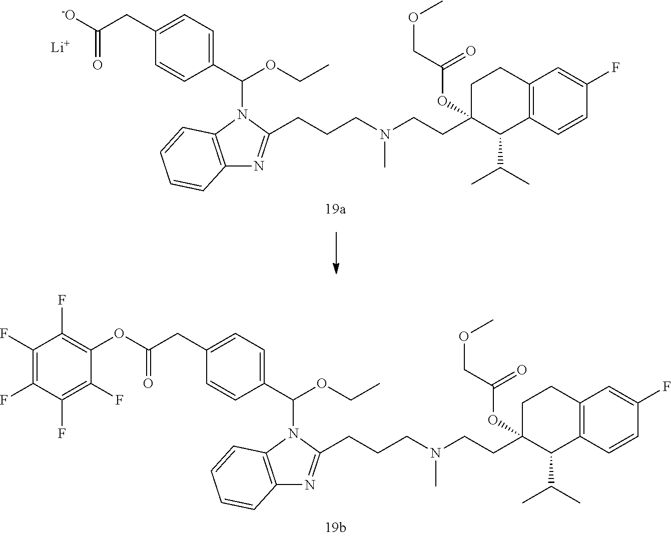

To a solution of compound 18b (1.0 eq) in THF/water (2:1) is added LiOH (1.2 eq), and the mixture is stirred at rt. The solvent is removed in vacuo to give 19a as the lithium salt. Compound 19a (1.0 eq) is combined with bis(pentafluorophenyl)carbonate (2.0 eq) in DMF. To the mixture is added DIPEA (6.0 eq) before stirring for 2 h. The solvent is removed in vacuo. Purification is performed via flash chromatography to give 19b.

### Example 21: Synthesis of Compound 20

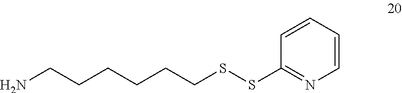 [SMILES: NCCCCCCSSc1ccccn1]

2,2′-dithiodipyridine (1.5 eq) is dissolved in anhydrous methanol and a solution of 6-amino-1-hexanethiol hydrochloride (1.0 eq) in anhydrous methanol is added dropwise. After complete addition the mixture is stirred at rt for 3 h. The solvent is removed in vacuo and the material is purified by flash chromatography to give 20.

### Example 22: Synthesis of Compound 21b

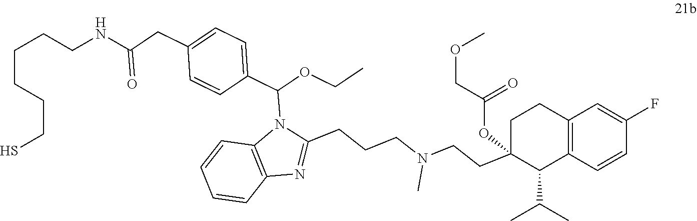 [SMILES: CCOC(c1ccc(CC(=O)NCCCCCCS)cc1)n1c(CCCN(C)CC[C@@]2(OC(=O)COC)CCc3cc(F)ccc3[C@@H]2C(C)C)nc2ccccc21]

To a solution of compound 19a (1 eq) in anhydrous DMF are added DIPEA (2.4 eq), PyBOP (1.2 eq) and compound 20 (1.2 eq) successively, and the reaction mixture is stirred at rt for 18 h. The solvent is removed in vacuo and the mixture is purified by flash chromatography to give 21a.

Compound 21a (1.0 eq) is dissolved in anhydrous DMF and a solution of tris(2-carboxyethyl)phosphine hydrochloride (4.0 eq) and DIPEA (4.0 eq) in DMSO is added. After complete addition the mixture is stirred at rt for 30 min. The solvent is removed in vacuo and the material is purified by flash chromatography to give 21b.

### Example 23: Synthesis of Compound 22

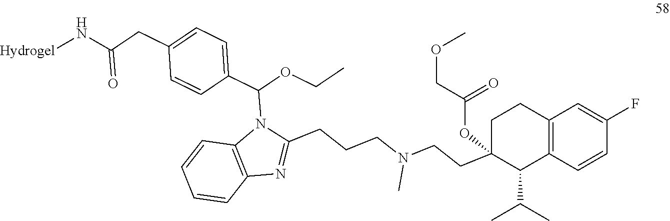

The amine-functionalized PEG-based hydrogel 5a is synthesized as described in WO 2011/012715 A1, Example 3. The hydrogel is swollen in 1% DIPEA in DMF in a syringe reactor with frit and washed three times with a 1% DIPEA/DMF solution. 19b (2.0 eq per hydrogel amine) is dissolved in 1% DIPEA in DMF. The solution is drawn into the hydrogel-containing reactor and shaken for 16 h at rt. The syringe is drained, the hydrogel washed several times with DMF, then washed several times with phosphate-buffered saline. A hydrogel suspension in pH 7.4 aqueous buffer is obtained.

### Example 24: Synthesis of Compound 23

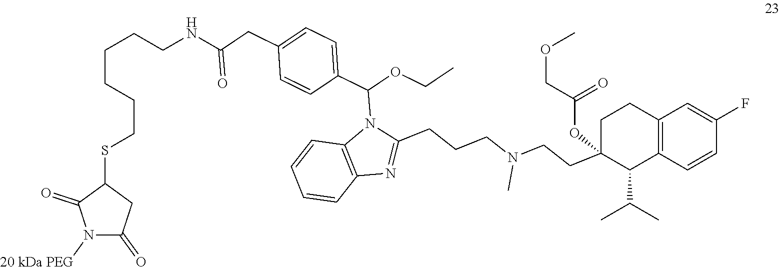

A solution of compound 21b (1.1 eq) in acetonitrile/water is mixed with PEG 20 kDa maleimide (1 eq) and the pH is adjusted to 7.0 by addition of pH 7.4 buffer (50 mM phosphate). The mixture is stirred at rt for 2 h and then purified by RP-HPLC purification without additional TFA to give compound 23.

### Example 25: Synthesis of Compound 24

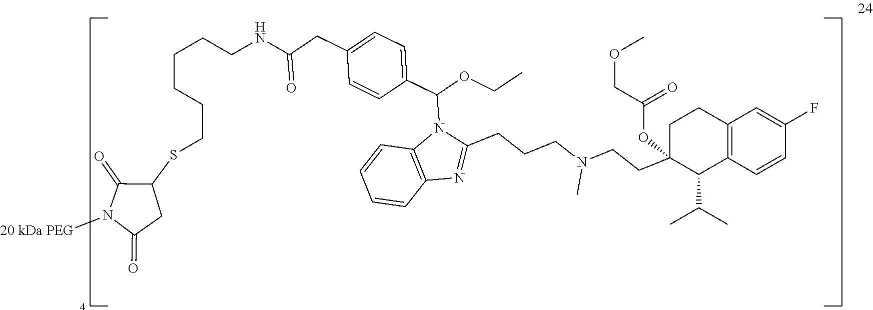

A solution of compound 21b (4.5 eq) in acetonitrile/water is mixed with 4-arm PEG 20 kDa maleimide (1 eq) and the pH is adjusted to 7.0 by addition of pH 7.4 buffer (50 mM phosphate). The mixture is stirred at rt for 2 h and then purified by RP-HPLC purification without additional TFA to give compound 24.

### Example 26: Synthesis of Compound 25

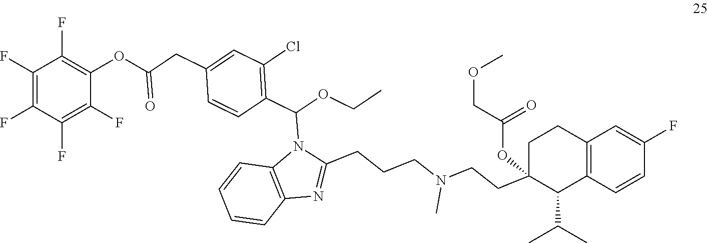 [SMILES: CCOC(c1ccc(CC(=O)Oc2c(F)c(F)c(F)c(F)c2F)cc1Cl)n1c(CCCN(C)CC[C@@]2(OC(=O)COC)CCc3cc(F)ccc3[C@@H]2C(C)C)nc2ccccc21]

The synthesis of compound 25 is performed according to the procedures described for example 19 and example 20, starting from compound 13.

### Example 27: Synthesis of Compound 26

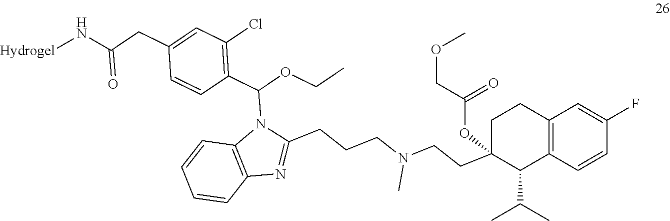

The synthesis of compound 26 is performed according to the procedure described for compound 22, using compound 25.

### Example 28: Synthesis of Compound 27

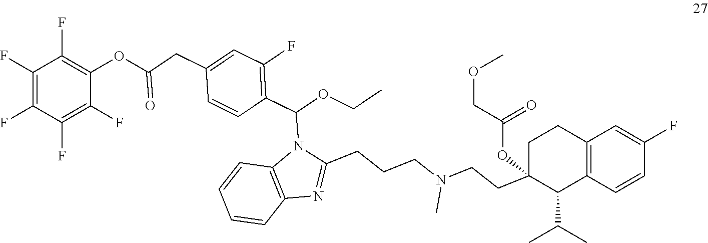 [SMILES: CCOC(c1ccc(CC(=O)Oc2c(F)c(F)c(F)c(F)c2F)cc1F)n1c(CCCN(C)CC[C@@]2(OC(=O)COC)CCc3cc(F)ccc3[C@@H]2C(C)C)nc2ccccc21]

The synthesis of compound 27 is performed according to the procedures described for example 19 and example 20, starting from compound 15.

### Example 29: Synthesis of Compound 28

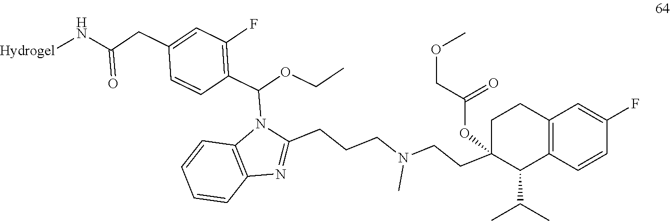

The synthesis of compound 28 is performed according to the procedure described for compound 22, using compound 27.

### Example 30: Synthesis of Compound 29

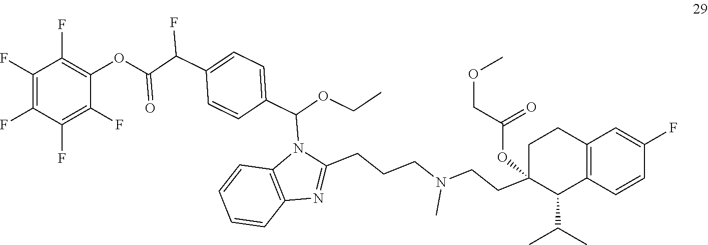 [SMILES: CCOC(c1ccc(C(F)C(=O)Oc2c(F)c(F)c(F)c(F)c2F)cc1)n1c(CCCN(C)CC[C@@]2(OC(=O)COC)CCc3cc(F)ccc3[C@@H]2C(C)C)nc2ccccc21]

The synthesis of compound 29 is performed according to the procedures described for example 19 and example 20, starting from compound 17.

### Example 31: Synthesis of Compound 30

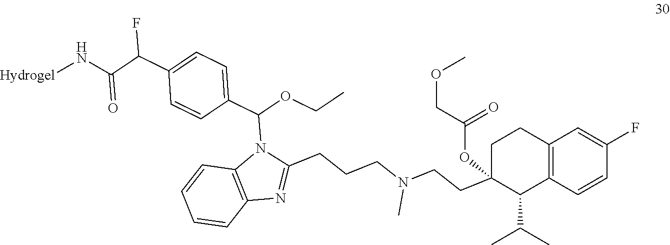

The synthesis of compound 30 is performed according to the procedure described for example 19 and example 20, using compound 29.

### Example 32: Release of Mibefradil In Vitro

Release of mibefradil from compounds 22, 23, 24, 26, 28 and 30 is effected by hydrolysis in 60 mM sodium phosphate buffer at pH 7.4 and 37° C. Unmodified mibefradil is released as assessed by LCMS.

### Abbreviations

DCM—dichloromethane
DIPEA—diisopropylethylamine
DMF—dimethylformamide
eq—equivalent
EtOH—ethanol
HPLC—high performance liquid chromatography
LCMS—liquid chromatography-coupled mass spectrometry
prep-HPLC—preparative high-performance liquid chromatography
PyBOP—benzotriazol-1-yl-oxytripyrrolidinophosphonium hexafluorophosphate
RP—reversed phase
rt—room temperature
THF—tetrahydrofuran
TsOH—para-toluene sulfonic acid

1. A conjugate or a pharmaceutically acceptable salt thereof comprising at least one moiety -D conjugated via at least one moiety -L¹-L²- to at least one moiety Z, wherein a moiety -L¹- is conjugated to a π-electron-pair-donating heteroaromatic N of a moiety -D and wherein the linkage between -D and -L¹- is reversible and wherein a moiety -L²- is conjugated to Z, wherein each -D is independently a π-electron-pair-donating heteroaromatic N-comprising moiety of a drug D-H;
each -L²- is independently a single bond or a spacer moiety;
each Z is independently a polymeric moiety or a C₈₋₂₄ alkyl;
each -L¹- is independently of formula (I): 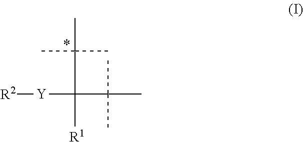
wherein
the dashed line marked with an asterisk indicates the attachment to -L²-;
the unmarked dashed line indicates the attachment to the π-electron-pair-donating heteroaromatic N of -D;
—Y— is selected from the group consisting of —N(R³)—, —O— and —S—;
R¹, —R² and —R³ are independently selected from the group consisting of —H, -T, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R⁴, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵)—, —OC(OR⁵)(R^{5a})—, —N(R⁵)C(O)N(R^{5a})— and —OC(O)N(R⁵)—;
each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each T is independently optionally substituted with one or more —R⁴, which are the same or different;
wherein —R⁴, —R⁵ and —R^{5a} are independently selected from the group consisting of —H and C₁₋₆ alkyl; wherein C₁₋₆ alkyl is optionally substituted with one or more halogen, which are the same or different; and
each -L¹- is substituted with -L²- and optionally further substituted.

2. The conjugate or pharmaceutically acceptable salt thereof of claim 1, wherein -D is selected from the group consisting of small molecule, medium size, peptide and protein drug moieties.

3. The conjugate or pharmaceutically acceptable salt thereof of claim 1 or 2, wherein -D is a small molecule drug moiety.

4. The conjugate or pharmaceutically acceptable salt thereof of any one of claims 1 to 3, wherein Z is a polymeric moiety.

5. The conjugate or pharmaceutically acceptable salt thereof of any one of claims 1 to 4, wherein Z is a water-insoluble polymeric moiety.

6. The conjugate or pharmaceutically acceptable salt thereof of any one of claims 1 to 5, wherein Z is a water-insoluble polymeric moiety comprising a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

7. The conjugate or pharmaceutically acceptable salt thereof of any one of claims 1 to 6, wherein Z is a hydrogel.

8. The conjugate or pharmaceutically acceptable salt thereof of any one of claims 1 to 7, wherein Z is a PEG-based or hyaluronic-acid based hydrogel.

9. The conjugate or pharmaceutically acceptable salt thereof of any one of claims 1 to 8, wherein Z is a PEG-based hydrogel.

10. The conjugate or pharmaceutically acceptable salt thereof of any one of claims 1 to 8, wherein Z is a hyaluronic-acid based hydrogel.

11. The conjugate or pharmaceutically acceptable salt thereof of any one of claims 1 to 4, wherein Z is a water-soluble polymeric moiety.

12. The conjugate or pharmaceutically acceptable salt thereof of any one of claims 1 to 11, wherein —Y— is —N(R³)—.

13. The conjugate or pharmaceutically acceptable salt thereof of any one of claims 1 to 12, wherein —R³ is —H.

14. The conjugate or pharmaceutically acceptable salt thereof of any one of claims 1 to 11, wherein —Y— is —O—.

15. The conjugate or pharmaceutically acceptable salt thereof of any one of claims 1 to 14, wherein —R¹ is —H.

16. The conjugate or pharmaceutically acceptable salt thereof of any one of claims 1 to 15, wherein —R² is C₁₋₆ alkyl.

17. The conjugate or pharmaceutically acceptable salt thereof of any one of claims 1 to 16, wherein -L²- is a spacer moiety.

18. The conjugate or pharmaceutically acceptable salt thereof of any one of claims 1 to 17, wherein -L²- has a molecular weight in the range of from 14 g/mol to 750 g/mol.

19. The conjugate or pharmaceutically acceptable salt thereof of any one of claims 1 to 18, wherein the linkage between Z and -L²- is stable.

20. The conjugate or pharmaceutically acceptable salt thereof of any one of claims 1 to 19, wherein -L²- is a spacer moiety selected from the group consisting of -T′-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T′-, C₁₋₅₀ alkyl, C₂₋₅₀ alkenyl and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T′-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR³)(R^{y3a})—, —N(R^{y3})C(O)N(R^{y3a})— and —OC(O)N(R^{y3})—;
wherein —R^{y1} and —R^{y1a} are independently selected from the group consisting of —H, -T′, C₁₋₅₀ alkyl, C₂₋₅₀ alkenyl and C₂₋₅₀ alkynyl; wherein -T′, C₁₋₅₀ alkyl, C₂₋₅₀ alkenyl, and C₂₋₅₀ alkynyl are optionally substituted with one or more —R^{y2}, which are the same or different, and wherein C₁₋₅₀ alkyl, C₂₋₅₀ alkenyl and C₂₋₅₀ alkynyl are optionally interrupted by one or more groups selected from the group consisting of -T′-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR⁴)(R^{y4a})—, —N(R^{y4})C(O)N(R^{y4a})—, and —OC(O)N(R^{y4})—;
each T′ is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl; wherein each T′ is independently optionally substituted with one or more —R^{y2}, which are the same or different;
each —R^{y2} is independently selected from the group consisting of halogen, —CN, oxo (═O), —COOR^{y5}, —OR^{y5}, —C(O)R^{y5}, —C(O)N(R⁵R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R⁵R^{y5a}) and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R^{y5}, —R^{y5a} and —R^{y5b} is independently selected from the group consisting of —H and C₁₋₆ alkyl; wherein C₁₋₆ alkyl is optionally substituted with one or more halogen, which are the same or different.

21. A pharmaceutical composition comprising the conjugate or pharmaceutically acceptable salt thereof of any one of claims 1 to 20.

22. The conjugate or pharmaceutically acceptable salt thereof of any one of claims 1 to 20 or the pharmaceutical composition of claim 21 for use as a medicament.

23. The conjugate or pharmaceutically acceptable salt thereof of any one of claims 1 to 20 or the pharmaceutical composition of claim 21 for use in a method of treating a disease that can be treated with D-H.

24. A method of preventing a disease or treating a patient suffering from a disease that can be prevented or treated with D-H, comprising administering an effective amount of the conjugate or the pharmaceutically acceptable salt thereof of any one of claims 1 to 20 or the pharmaceutical composition of claim 21 to the patient.
